(19) United States
(12) Patent Application Publication
(10) Pub. No.: US 2018/0263710 A1
(43) Pub. Date: Sep. 20, 2018

(54) MEDICAL IMAGING APPARATUS AND SURGICAL NAVIGATION SYSTEM

(71) Applicant: SONY CORPORATION, Tokyo (JP)

(72) Inventors: Tatsumi SAKAGUCHI, Kanagawa (JP); Takara KASAI, Kanagawa (JP)

(73) Assignee: SONY CORPORATION, Tokyo (JP)

(21) Appl. No.: 15/761,507

(22) PCT Filed: Nov. 18, 2016
(86) PCT No.: PCT/JP2016/084354

§ 371 Date: Mar. 20, 2018

(30) Foreign Application Priority Data
Dec. 25, 2015 (JP) 2015-252869

(51) Int. Cl. A61B 34/20; A61B 90/20; A61B 34/00; A61B 90/00

(52) U.S. Cl. CPC A61B 34/20; A61B 90/20; A61B 90/50; A61B 90/37; A61B 34/70

(57) ABSTRACT

A surgical information processing apparatus, including circuitry that obtains position information of a surgical imaging device, the position information indicating displacement of the surgical imaging device from a predetermined position, in a registration mode, obtain first image information from the surgical imaging device regarding a position of a surgical component, determines the position of the surgical component based on the first image information and the position information, and in an imaging mode, obtains second image information from the surgical imaging device of the surgical component based on the determined position.

### CROSS REFERENCE TO RELATED APPLICATIONS

This application claims the benefit of Japanese Priority Patent Application JP 2015-252869 filed Dec. 25, 2015, the entire contents of which are incorporated herein by reference.

### TECHNICAL FIELD

The present disclosure relates to a surgical information processing apparatus and method.

### BACKGROUND ART

Thus far, surgical navigation systems for assisting accurate operations have been known. The surgical navigation system is used in the field of, for example, neurosurgery, otolaryngology, orthopedics, or the like; and displays an image in which an MRI image, a 3D model, or the like prepared in advance is superimposed on a captured image of a surgical field, and thus assists an operation so that the operation is advanced in accordance with a prior plan. Such a surgical navigation system includes, for example, a position detection device for detecting the position of a microscope, a patient, or a surgical instrument. That is, since neither the microscope nor the surgical instrument has a section for acquiring the relationship between the relative three-dimensional positions of the microscope or the surgical instrument itself and the patient, a section for finding the mutual positional relationship is necessary.

As such a position detection device, for example, a device using an optical marker and an optical sensor is known. In PTL 1, a section for detecting the position and posture of a rigid scope which is composed of a position sensor formed of a photodetector such as a CCD camera, a light emitting unit provided at the rigid scope as a surgical instrument and formed of a light source such as an LED, and a position calculation unit is disclosed.

### CITATION LIST

### Patent Literature

PTL 1: JP 2002-102249A

### SUMMARY

### Technical Problem

However, in the optical position detection device disclosed in PTL 1, when a physical shield is present between the light emitting unit provided at the rigid scope and the optical sensor, position detection may be no longer possible. For example, there are many surgical instruments and surgical staff members in the surgical place; hence, to prevent a physical shield between the light emitting unit and the optical sensor, an inconvenience such as the necessity to install the optical sensor in a high position may occur.

Other than the optical position detection device, there is a magnetic field-type position detection device using a magnetic field generating device and a magnetic sensor; but in the magnetic field-type position detection device, when an electrically conductive device or the like is used in a device other than the magnetic field generating device for position detection or a surgical instrument, the detection result may have an error, or it may be difficult to perform position detection. Furthermore, also in the magnetic field-type position detection device, similarly to the optical position detection device, position detection may be no longer possible when a physical shield is present between the magnetic field generating device and the magnetic sensor.

### Solution to Problem

According to the present disclosure, there is provided a surgical information processing apparatus, including circuitry that obtains position information of a surgical imaging device, the position information indicating displacement of the surgical imaging device from a predetermined position, in a registration mode, obtain first image information from the surgical imaging device regarding a position of a surgical component, determines the position of the surgical component based on the first image information and the position information, and in an imaging mode, obtains second image information from the surgical imaging device of the surgical component based on the determined position.

Further, according to the present disclosure, there is provided a surgical information processing method implemented using circuitry, including the steps of obtaining first position information of a surgical imaging device, the first position information indicating displacement of the surgical imaging device from a predetermined position, generating second position information of a surgical component with respect to the surgical imaging device based on first image information obtained in a registration mode from the surgical imaging device, determining the position of a surgical component with respect to the predetermined position based on first position information and the second position information, and in an imaging mode, obtaining second image information from the medical imaging device of the surgical component based on the determined position.

Further, according to the present disclosure, there is provided a non-transitory computer readable medium having stored therein a program that when executed by a computer including circuitry causes the computer to implement a surgical information processing method implemented using circuitry, including the steps of obtaining first position information of a surgical imaging device, the first position information indicating displacement of the surgical imaging device from a predetermined position, generating second position information of a surgical component with respect to the surgical imaging device based on first image information obtained in a registration mode from the surgical imaging device, determining the position of a surgical component with respect to the predetermined position based on first position information and the second position information, and in an imaging mode, obtaining second image information from the medical imaging device of the surgical component based on the determined position.

### Advantageous Effects of Invention

As described above, according to an embodiment of the present disclosure, a medical imaging apparatus and a surgical navigation system capable of calculating a predetermined position on the basis of information acquired by an imaging apparatus that images a patient, without using an additional sensor such as an optical sensor or a magnetic sensor, can be obtained. Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an illustration diagram for describing a rough configuration of a surgical navigation system including an imaging apparatus.

FIG. 2 is an illustration diagram showing an example of the configuration of the imaging apparatus.

FIG. 3 is a block diagram showing an example of the system configuration of the surgical navigation system including the imaging apparatus.

FIG. 4 is a block diagram showing the functional configuration of a position computation unit of the imaging apparatus.

FIG. 5 is an illustration diagram showing an example of the use of the surgical navigation system including the imaging apparatus.

FIG. 6 is an illustration diagram showing a situation of an operation for which a surgical navigation system according to a first embodiment of the present disclosure can be used.

FIG. 7 is a flow chart showing the processing of grasping a surgical field of the surgical navigation system according to the embodiment.

FIG. 8 is a flow chart showing the processing of grasping a surgical field of the surgical navigation system according to the embodiment.

FIG. 9 is a flow chart showing the registration processing of the surgical navigation system according to the embodiment.

FIG. 10 is a flow chart showing the automatic registration processing of the surgical navigation system according to the embodiment.

FIG. 11 is a flow chart showing the processing of detecting the position of the tip of a surgical instrument of the surgical navigation system according to the embodiment.

FIG. 12 is a flow chart showing the processing of detecting the position of the tip of a surgical instrument of the surgical navigation system according to the embodiment.

FIG. 13 is an illustration diagram showing an example of the configuration of an imaging apparatus according to a second embodiment of the present disclosure.

FIG. 14 is an illustration diagram showing a situation of an operation for which a surgical navigation system according to the embodiment can be used.

FIG. 15 is a flow chart showing the registration processing of the surgical navigation system according to the embodiment.

FIG. 16 is a flow chart showing the processing of detecting the position of the tip of a surgical instrument of the surgical navigation system according to the embodiment.

FIG. 17 is a flow chart showing the processing of examining the positional shift of a stereo camera by the imaging apparatus according to the embodiment.

FIG. 18 is a flow chart showing the recalibration processing by the imaging apparatus according to the embodiment.

### DESCRIPTION OF EMBODIMENTS

Hereinafter, (a) preferred embodiment(s) of the present disclosure will be described in detail with reference to the appended drawings. In this specification and the appended drawings, structural elements that have substantially the same function and structure are denoted with the same reference numerals, and repeated explanation of these structural elements is omitted.

The description is given in the following order.

1. Basic configuration of the surgical navigation system

1-1. Examples of the configuration of the surgical navigation system

1-2. Examples of the system configuration of the surgical navigation system

1-3. Examples of the use of the surgical navigation system

2. First embodiment (an example using a bed-mounted arm)

2-1. Overview of the surgical navigation system

2-2. Control processing

2-3. Conclusions

3. Second embodiment (an example using an arm movable cart)

3-1. Overview of the surgical navigation system

3-2. Control processing

3-3. Conclusions

In the following description, “the user” refers to any medical staff member who uses the imaging apparatus or the surgical navigation system, such as an operator or an assistant.

### 1. Basic Configuration of the Surgical Navigation System

First, the basic configuration common to the embodiments described later out of the configuration of an imaging apparatus to which the technology according to the present disclosure can be applied or a surgical navigation system including the imaging apparatus is described.

<1-1. Examples of the Configuration of the Surgical Navigation System>

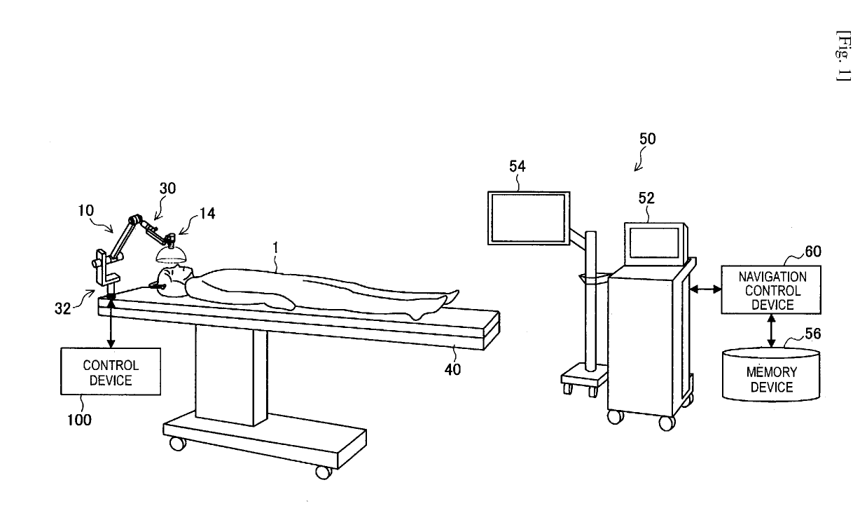

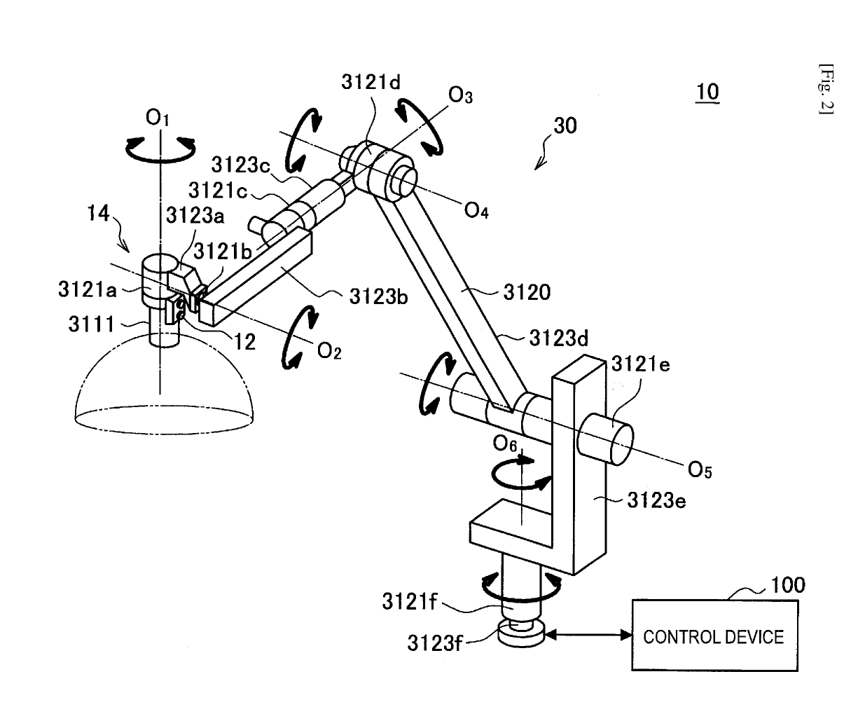

FIG. 1 is an illustration diagram for describing a rough configuration of a surgical navigation system. FIG. 2 is an illustration diagram showing an example of the configuration of an imaging apparatus 10. The surgical navigation system includes an imaging apparatus 10 that images an object to be observed (a surgical site of a patient 1) and a navigation apparatus 50 that performs the navigation of an operation using a surgical field image captured by the imaging apparatus 10. The surgical navigation system is a system for assisting an operator so that an operation is advanced in accordance with a prior plan. An image in which a preoperative image or a 3D model of the surgical site that is prepared in advance and includes the information of the position of incision, the position of an affected part, a treatment procedure, etc. is superimposed on a surgical field image captured by the imaging apparatus 10 may be displayed on a display device 54 of the navigation apparatus 50.

(1-1-1. Imaging Apparatus)

The imaging apparatus 10 includes a microscope unit 14 for imaging the surgical site of the patient 1 and an arm unit 30 that supports the microscope unit 14. The microscope unit 14 corresponds to a camera in the technology of an embodiment of the present disclosure, and is composed of an imaging unit (not illustrated) provided in a cylindrical unit 3111 in a substantially circular cylindrical shape and a manipulation unit (hereinafter, occasionally referred to as a “camera manipulation interface”) 12 provided in a partial area of the outer periphery of the cylindrical unit 3111. The microscope unit 14 is an electronic imaging microscope unit (what is called a video microscope unit) that electronically acquires a captured image with the imaging unit.

A cover glass that protects the imaging unit provided inside is provided on the opening surface at the lower end of the cylindrical unit 3111. The light from the object to be observed (hereinafter, occasionally referred to as observation light) passes through the cover glass, and is incident on the imaging unit in the cylindrical unit 3111. A light source formed of, for example, a light emitting diode (LED) or the like may be provided in the cylindrical unit 3111, and at the time of imaging, light may be applied from the light source to the object to be observed via the cover glass.

The imaging unit is composed of an optical system that collects observation light and an imaging element that receives the observation light collected by the optical system. The optical system is configured such that a plurality of lenses including a zoom lens and a focus lens are combined, and the optical characteristics thereof are adjusted so as to cause observation light to form an image on the light receiving surface of the imaging element. The imaging element receives and photoelectrically converts observation light, and thereby generates a signal corresponding to the observation light, that is, an image signal corresponding to the observed image. As the imaging element, for example, an imaging element having the Bayer arrangement to allow color photographing is used. The imaging element may be any of various known imaging elements such as a complementary metal oxide semiconductor (CMOS) image sensor and a charge-coupled device (CCD) image sensor.

The image signal generated by the imaging element is transmitted as raw data to a not-illustrated control device 100. Here, the transmission of the image signal may preferably be performed by optical communication. This is because in the surgical place the operator performs an operation while observing the condition of an affected part using a captured image, and therefore for a safer and more reliable operation it is required that moving images of the surgical site be displayed in real time to the extent possible. By the image signal being transmitted by optical communication, the captured image can be displayed with low latency.

The imaging unit may include a driving mechanism that moves the zoom lens and the focus lens of the optical system along the optical axis. By the zoom lens and the focus lens being moved as appropriate by the driving mechanism, the magnification of the captured image and the focal distance at the time of imaging can be adjusted. In the imaging unit, also various functions that may be generally provided in an electronic imaging microscope unit, such as an auto-exposure (AE) function and an auto-focus (AF) function, may be mounted.

The imaging unit may be configured as what is called a single-chip imaging unit including one imaging element, or may be configured as what is called a multi-chip imaging unit including a plurality of imaging elements. In the case where the imaging unit is configured as a multi-chip type, for example, an image signal corresponding to each of RGB may be generated by each imaging element, and the image signals thus generated may be synthesized to obtain a color image. Alternatively, the imaging unit may be configured so as to include a pair of imaging elements for acquiring image signals for the right eye and the left eye, respectively, corresponding to stereoscopic vision (3D display). In this case, the microscope unit 14 is configured as a stereo camera. By 3D display being performed, the operator can grasp the depth of the surgical site more accurately. The imaging apparatus 10 of each embodiment according to the present disclosure includes a stereo camera as the microscope unit 14. In the case where the imaging unit is configured as a multi-chip type, a plurality of optical systems may be provided to correspond to the imaging elements.

The camera manipulation interface 12 is formed of, for example, a cross lever, a switch, or the like, and is an input section that receives the manipulation input of the user. For example, the user may input, via the camera manipulation interface 12, instructions to alter the magnification of the observed image and the focal distance to the object to be observed. The driving mechanism of the imaging unit may move the zoom lens and the focus lens as appropriate in accordance with the instructions, and thereby the magnification and the focal distance can be adjusted. Furthermore, for example, the user may input, via the camera manipulation interface 12, an instruction to switch the operating mode of the arm unit 30 (an all-free mode and a fixed mode described later).

When the user intends to move the microscope unit 14, the user may move the microscope unit 14 in a state of grasping the cylindrical unit 3111 by gripping it. In this case, in order that the camera manipulation interface 12 can be manipulated even while the user moves the cylindrical unit 3111, the camera manipulation interface 12 may be provided in a position where the user can easily manipulate it with the finger in the state of gripping the cylindrical unit 3111. Alternatively, the user may manipulate an input device (hereinafter, occasionally referred to as an “arm manipulation interface”) to control the posture of the arm unit 30 to move the microscope unit 14.

The arm unit 30 is configured by a plurality of links (a first link 3123a to a sixth link 31230 being linked together in a rotationally movable manner relative to each other by a plurality of joint units (a first joint unit 3121a to a sixth joint unit 31210.

The first joint unit 3121a has a substantially circular columnar shape, and supports, at its tip (its lower end), the upper end of the cylindrical unit 3111 of the microscope unit 14 in a rotationally movable manner around a rotation axis (a first axis O₁) parallel to the center axis of the cylindrical unit 3111. Here, the first joint unit 3121a may be configured such that the first axis O₁ coincides with the optical axis of the imaging unit of the microscope unit 14. Thereby, the microscope unit 14 can be rotationally moved around the first axis O₁, and thus the visual field can be altered so as to rotate the captured image.

The first link 3123a fixedly supports, at its tip, the first joint unit 3121a. Specifically, the first link 3123a is a bar-like member having a substantially L-shaped configuration, and is connected to the first joint unit 3121a in such a manner that one side on the tip side of the first link 3123a extends in a direction orthogonal to the first axis O₁ and the end of the one side is in contact with an upper end portion of the outer periphery of the first joint unit 3121a. The second joint unit 3121b is connected to the end of the other side on the root end side of the substantially L-shaped configuration of the first link 3123a.

The second joint unit 3121b has a substantially circular columnar shape, and supports, at its tip, the root end of the first link 3123a in a rotationally movable manner around a rotation axis (a second axis O₂) orthogonal to the first axis O₁. The tip of the second link 3123b is fixedly connected to the root end of the second joint unit 3121b.

The second link 3123b is a bar-like member having a substantially L-shaped configuration, and one side on its tip side extends in a direction orthogonal to the second axis O₂ and the end of the one side is fixedly connected to the root end of the second joint unit 3121b. The third joint unit 3121c is connected to the other side on the root end side of the substantially L-shaped configuration of the second link 3123b.

The third joint unit 3121c has a substantially circular columnar shape, and supports, at its tip, the root end of the second link 3123b in a rotationally movable manner around a rotation axis (a third axis O₃) orthogonal to both of the first axis O₁ and the second axis O₂. The tip of the third link 3123c is fixedly connected to the root end of the third joint unit 3121c. By rotationally moving the formation on the tip side including the microscope unit 14 around the second axis O₂ and the third axis O₃, the microscope unit 14 can be moved so that the position of the microscope unit 14 in the horizontal plane is altered. In other words, by controlling the rotation around the second axis O₂ and the third axis O₃, the visual field of the captured image can be moved in the plane.

The third link 3123c is configured such that the tip side has a substantially circular columnar shape, and the root end of the third joint unit 3121c is fixedly connected to the tip of the circular columnar shape in such a manner that both have substantially the same center axis. The root end side of the third link 3123c has a prismatic shape, and the fourth joint unit 3121d is connected to the end on the root end side.

The fourth joint unit 3121d has a substantially circular columnar shape, and supports, at its tip, the root end of the third link 3123c in a rotationally movable manner around a rotation axis (a fourth axis O₄) orthogonal to the third axis O₃. The tip of the fourth link 3123d is fixedly connected to the root end of the fourth joint unit 3121d.

The fourth link 3123d is a bar-like member extending substantially in a straight line, and extends orthogonally to the fourth axis O₄ and is fixedly connected to the fourth joint unit 3121d in such a manner that the end of the tip of the fourth link 3123d is in contact with a side surface of the substantially circular columnar shape of the fourth joint unit 3121d. The fifth joint unit 3121e is connected to the root end of the fourth link 3123d.

The fifth joint unit 3121e has a substantially circular columnar shape, and supports, on its tip side, the root end of the fourth link 3123d in a rotationally movable manner around a rotation axis (a fifth axis O₅) parallel to the fourth axis O₄. The tip of the fifth link 3123e is fixedly connected to the root end of the fifth joint unit 3121e. The fourth axis O₄ and the fifth axis O₅ are rotation axes that allow the microscope unit 14 to move in the vertical direction. By rotationally moving the formation on the tip side including the microscope unit 14 around the fourth axis O₄ and the fifth axis O₅, the height of the microscope unit 14, that is, the distance between the microscope unit 14 and the object to be observed can be adjusted.

The fifth link 3123e is configured such that a first member having a substantially L-shaped configuration in which one side extends in the vertical direction and the other side extends in the horizontal direction and a second member in a bar-like shape that extends downward in the vertical direction from the portion extending in the horizontal direction of the first member are combined. The root end of the fifth joint unit 3121e is fixedly connected to the vicinity of the upper end of the portion extending in the vertical direction of the first member of the fifth link 3123e. The sixth joint unit 3121f is connected to the root end (the lower end) of the second member of the fifth link 3123e.

The sixth joint unit 3121f has a substantially circular columnar shape, and supports, on its tip side, the root end of the fifth link 3123e in a rotationally movable manner around a rotation axis (a sixth axis O₆) parallel to the vertical direction. The tip of the sixth link 3123f is fixedly connected to the root end of the sixth joint unit 3121f.

The sixth link 3123f is a bar-like member extending in the vertical direction, and its root end is fixedly connected to the upper surface of a bed 40.

The range in which the first joint unit 3121a to the sixth joint unit 3121f can rotate is appropriately set so that the microscope unit 14 can make desired movements. Thereby, in the arm unit 30 having the configuration described above, movements with 3 degrees of freedom of translation and 3 degrees of freedom of rotation, i.e. a total of 6 degrees of freedom, can be achieved for the movement of the microscope unit 14. By thus configuring the arm unit 30 so that 6 degrees of freedom are achieved for the movement of the microscope unit 14, the position and posture of the microscope unit 14 can be freely controlled in the range in which the arm unit 30 can move. Therefore, the surgical site can be observed from any angle, and the operation can be executed more smoothly.

The illustrated configuration of the arm unit 30 is only an example, and the number and shape (length) of links and the number, arrangement position, direction of the rotation axis, etc. of joint units that constitute the arm unit 30 may be appropriately designed so that desired degrees of freedom can be achieved. For example, although as described above it is preferable that the arm unit 30 be configured to have 6 degrees of freedom in order to freely move the microscope unit 14, the arm unit 30 may be configured to have larger degrees of freedom (that is, redundant degrees of freedom). In the case where there are redundant degrees of freedom, the posture of the arm unit 30 can be altered in a state where the position and posture of the microscope unit 14 are fixed. Thus, control with higher convenience for the operator can be achieved, such as controlling the posture of the arm unit 30 so that the arm unit 30 does not interfere with the visual field of the operator who views the display device 54 of the navigation apparatus 50.

Here, the first joint unit 3121a to the sixth joint unit 3121f may be provided with a driving mechanism such as a motor and an actuator equipped with an encoder or the like that detects the rotation angle in each joint unit. The driving of each actuator provided in the first joint unit 3121a to the sixth joint unit 3121f may be controlled as appropriate by the control device 100, and thereby the posture of the arm unit 30, that is, the position and posture of the microscope unit 14 can be controlled. The value detected by the encoder provided in each joint unit may be used as posture information concerning the posture of the arm unit 30.

Further, the first joint unit 3121a to the sixth joint unit 3121f may be provided with a brake that restricts the rotation of the joint unit. The operation of the brake may be controlled by the control device 100. For example, when it is intended to fix the position and posture of the microscope unit 14, the control device 100 puts the brake of each joint unit into operation. Thereby, the posture of the arm unit 30, that is, the position and posture of the microscope unit 14 can be fixed without driving the actuator, and therefore the power consumption can be reduced. When it is intended to move the position and posture of the microscope unit 14, the control device 100 may release the brake of each joint unit, and may drive the actuator in accordance with a predetermined control system.

Such an operation of the brake may be performed in accordance with the manipulation input by the user via the camera manipulation interface 12 described above. When the user intends to move the position and posture of the microscope unit 14, the user manipulates the camera manipulation interface 12 to release the brake of each joint unit. Thereby, the operating mode of the arm unit 30 transitions to a mode in which the rotation in each joint unit can be freely made (an all-free mode). Further, when the user intends to fix the position and posture of the microscope unit 14, the user manipulates the camera manipulation interface 12 to put the brake in each joint unit into operation. Thereby, the operating mode of the arm unit 30 transitions to a mode in which the rotation in each joint unit is restricted (a fixed mode).

The control device 100 puts the actuator of the first joint unit 3121a to the sixth joint unit 3121f into operation in accordance with a predetermined control system, and thereby controls the driving of the arm unit 30. Further, for example, the control device 100 controls the operation of the brake of the first joint unit 3121a to the sixth joint unit 3121f, and thereby alters the operating mode of the arm unit 30.

Further, the control device 100 outputs an image signal acquired by the imaging unit of the microscope unit 14 of the imaging apparatus 10 to the navigation apparatus 50. At this time, the control device 100 outputs also the information of the position of the surgical site of the patient 1 and the position of a surgical instrument to the navigation apparatus 50.

(1-1-2. Navigation Apparatus)

The navigation apparatus 50 includes a navigation manipulation interface 52 through which the manipulation input of the navigation apparatus 50 is performed by the user, the display device 54, a memory device 56, and a navigation control device 60. The navigation control device 60 performs various signal processings on an image signal acquired from the imaging apparatus 10 to produce 3D image information for display, and causes the display device 54 to display the 3D image information. In the signal processings, various known signal processings such as development processing (demosaic processing), image quality improvement processing (range enhancement processing, super-resolution processing, noise reduction (NR) processing, camera shake compensation processing, and/or the like), and/or magnification processing (i.e. electronic zoom processing) may be performed.

The navigation apparatus 50 is provided in the operating room, and displays an image corresponding to 3D image information produced by the navigation control device 60 on the display device 54, on the basis of a control command of the navigation control device 60. The navigation control device 60 corresponds to a navigation control unit in the technology of an embodiment of the present disclosure. On the display device 54, an image of the surgical site photographed by the microscope unit 14 may be displayed. The navigation apparatus 50 may cause the display device 54 to display, in place of or together with an image of the surgical site, various pieces of information concerning the operation such as the information of the body of the patient 1 and/or information regarding the surgical technique. In this case, the display of the display device 54 may be switched as appropriate by the user's manipulation. Alternatively, a plurality of display devices 54 may be provided, and an image of the surgical site and various pieces of information concerning the operation may be displayed individually on the plurality of display devices 54. As the display device 54, various known display devices such as a liquid crystal display device or an electro-luminescence (EL) display device may be used.

In the memory device 56, for example, a preoperative image or a 3D model of the surgical site of the patient 1 of which the relative relationship with a predetermined reference position in the three-dimensional space is found in advance is stored. For example, prior to the operation, a preoperative image is produced or a 3D model of the surgical site is produced on the basis of an MRI image or the like of a part including the surgical site of the patient 1. Then, information for assisting the operation such as the position of incision, the position of an affected part, and the position of excision may be superimposed on the preoperative image or the 3D model, or on an image of contours or the like of the surgical site of the patient 1 obtained from the preoperative image or the 3D model, and the resulting image may be stored in the memory device 56. The navigation control device 60 superimposes at least one preoperative image or 3D model on 3D image information captured by the microscope unit 14 to produce 3D image information, and causes the display device 54 to display the 3D image information. The memory device 56 may be provided in the navigation apparatus 50, or may be provided in a server connected via a network or the like.

<1-2. Examples of the System Configuration of the Surgical Navigation System>

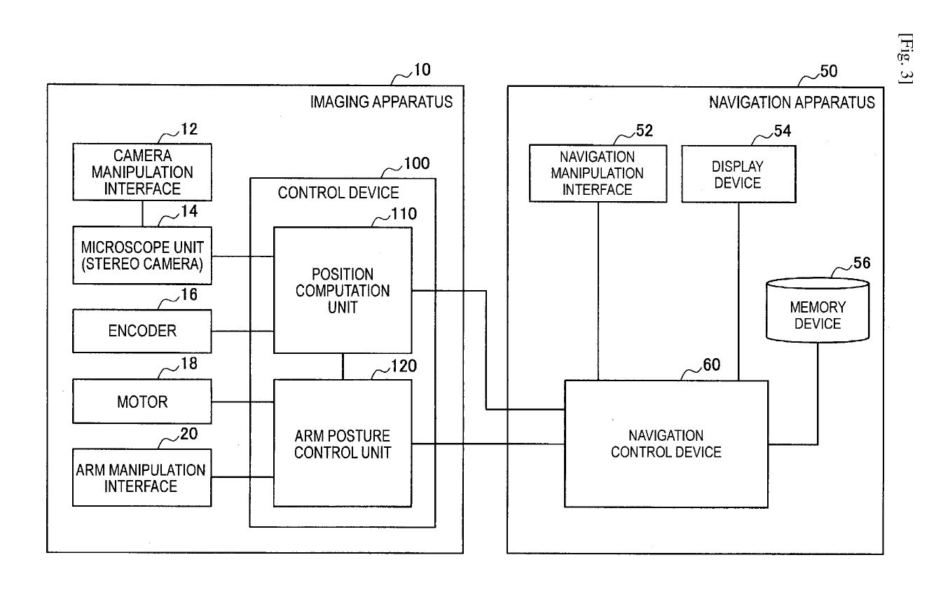

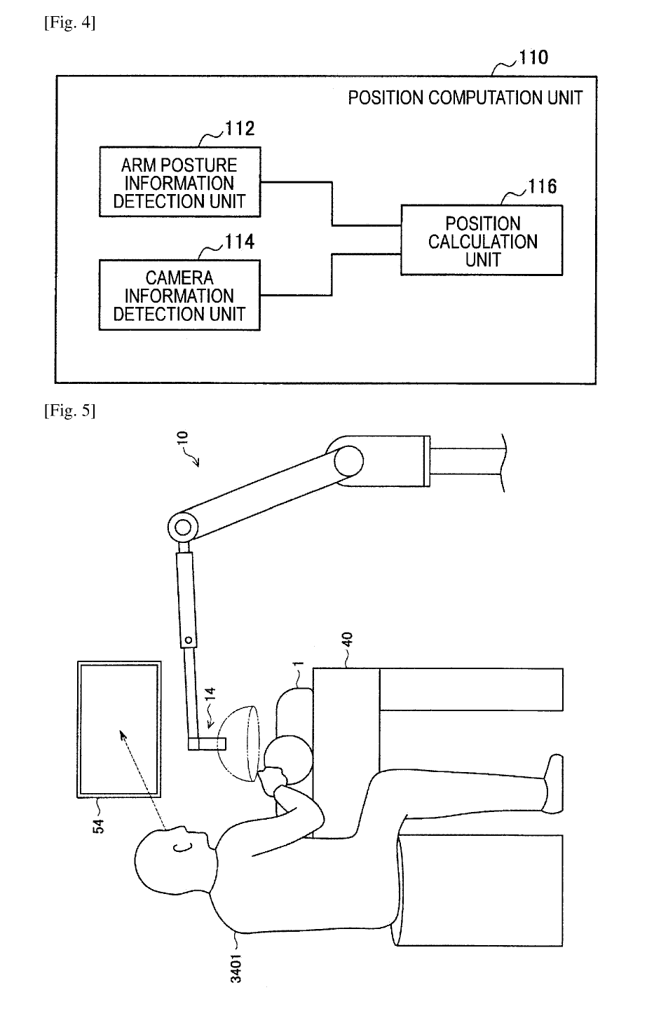

FIG. 3 is a block diagram showing an example of the system configuration of the surgical navigation system. FIG. 4 is a block diagram showing the functional configuration of a position computation unit 110 of the control device 100. The imaging apparatus 10 includes the camera manipulation interface 12, the microscope unit 14, an encoder 16, a motor 18, an arm manipulation interface 20, and the control device 100. Of them, the encoder 16 and the motor 18 are mounted on the actuator provided in the joint unit of the arm unit 30. The navigation apparatus 50 includes the navigation manipulation interface 52, the display device 54, the memory device 56, and the navigation control device 60.

The control device 100 may be a processor such as a central processing unit (CPU) or a graphics processing unit (GPU), or a microcomputer, a control board, or the like in which a processor and a memory element such as a memory are combined. The processor of the control device 100 operates in accordance with a predetermined program, and thereby the various functions described above can be achieved. Although in the illustrated example the control device 100 is provided as a separate device from the imaging apparatus 10, the control device 100 may be installed in the imaging apparatus 10 and may be configured integrally with the imaging apparatus 10. Alternatively, the control device 100 may be composed of a plurality of devices. For example, a microcomputer, a control board, or the like may be provided in each of the microscope unit 14 and the first joint unit 3121a to the sixth joint unit 3121f of the arm unit 30, and they may be connected to be communicable with each other; thereby, a similar function to the control device 100 can be achieved.

Similarly, also the navigation control device 60 may be a processor such as a CPU or a GPU, or a microcomputer, a control board, or the like in which a processor and a memory element such as a memory are combined. The processor of the navigation control device 60 operates in accordance with a predetermined program, and thereby the various functions described above can be achieved. Although in the illustrated example the navigation control device 60 is provided as a separate device from the navigation apparatus 50, the navigation control device 60 may be installed in the navigation apparatus 50 and may be configured integrally with the navigation apparatus 50. Alternatively, the navigation control device 60 may be composed of a plurality of devices.

The communication between the control device 100 and the microscope unit 14 and the communication between the control device 100 and the first joint unit 3121a to the sixth joint unit 3121f may be wired communication or may be wireless communication. The communication between the navigation control device 60 and the navigation manipulation interface 52, the communication between the navigation control device 60 and the display device 54, and the communication between the navigation control device 60 and the memory device 56 may be wired communication or may be wireless communication. In the case of wired communication, communication by electrical signals may be performed, or optical communication may be performed. In this case, the transmission cable used for the wired communication may be configured as an electrical signal cable, an optical fiber, or a composite cable of these in accordance with the communication system. On the other hand, in the case of wireless communication, since it is not necessary to lay transmission cables in the operating room, a situation in which the movements of medical staff members in the operating room are hindered by such transmission cables can be avoided.

The control device 100 of the imaging apparatus 10 includes a position computation unit 110 and an arm posture control unit 120. The position computation unit 110 calculates a predetermined position on the basis of information acquired from the microscope unit 14 and information acquired from the encoder 16. The position computation unit 110 transmits the calculation result to the navigation control device 60. A design in which the calculation result obtained by the position computation unit 110 is readable by the arm posture control unit 120 is possible. Further, the position computation unit 110 outputs image information based on an image signal acquired by the microscope unit 14 to the navigation control device 60. In this case, the position computation unit 110 corresponds also to an output unit that outputs image information produced from an image signal acquired by the microscope unit 14.

As shown in FIG. 4, the position computation unit 110 includes an arm posture information detection unit 112, a camera information detection unit 114, and a position calculation unit 116. The arm posture information detection unit 112 grasps the current posture of the arm unit 30 and the current position and posture of the microscope unit 14 on the basis of information concerning the rotation angle of each joint unit detected by the encoder 16. The camera information detection unit 114 acquires image information concerning an image captured by the microscope unit 14. In the image information acquired, also the information of the focal distance and magnification of the microscope unit 14 may be included. The focal distance of the microscope unit 14 may be outputted while being replaced with, for example, the distance from the rotation axis of the second joint unit 3121b that supports the microscope unit 14 in the arm unit 30 to the surgical site of the patient 1. The processing executed by the position computation unit 110 will be described in detail in the later embodiments.

Returning to FIG. 3, the arm posture control unit 120 drives the motor 18 provided in each joint unit of the arm unit 30 on the basis of a control command from the navigation control device 60, and thus controls the arm unit 30 to a predetermined posture. Thereby, for example, the surgical site of the patient 1 can be imaged from a desired angle by the microscope unit 14. The arm posture control unit 120 may control each motor 18 on the basis of the calculation result of the position computation unit 110.

Specifically, using the posture information of the arm unit 30 detected by the position computation unit 110, the arm posture control unit 120 calculates a control value for each joint unit (for example, the rotation angle, the torque to be generated, etc.) which achieves a movement of the microscope unit 14 in accordance with the manipulation input from the user or the control command from the navigation control device 60. The arm posture control unit 120 drives the motor 18 of each joint unit in accordance with the calculated control value. At this time, the system of the control of the arm unit 30 by the arm posture control unit 120 is not limited, and various known control systems such as force control or position control may be employed.

For example, the operator may perform a manipulation input via a not-illustrated arm manipulation interface 20 as appropriate; thereby, the driving of the arm unit 30 can be appropriately controlled by the arm posture control unit 120 in accordance with the manipulation input, and the position and posture of the microscope unit 14 can be controlled. By the control, the microscope unit 14 can be moved from an arbitrary position to an arbitrary position and then fixedly supported at the position after the movement. As the arm manipulation interface 20, one that can be manipulated even when the operator holds a surgical instrument in the hand, such as a foot switch, is preferably used in view of the convenience of the operator. The manipulation input may be performed in a non-contact manner based on gesture tracking or eye-gaze tracking using a wearable device or a camera provided in the operating room. Thereby, even a user in a clean area can manipulate a device in an unclean area with higher degrees of freedom. Alternatively, the arm unit 30 may be manipulated by what is called a master-slave system. In this case, the arm unit 30 may be remotely manipulated by the user via the arm manipulation interface 20 installed in a place distant from the operating room.

Further, in the case where force control is employed, what is called power-assisted control may be performed in which an external force from the user is received and the motor 18 of the first joint unit 3121a to the sixth joint unit 3121f is driven so that the arm unit 30 moves smoothly in accordance with the external force. Thus, when the user intends to directly move the position of the microscope unit 14 by grasping it, the user can move the microscope unit 14 with a relatively small force. Therefore, the microscope unit 14 can be moved more intuitively by a simpler manipulation, and the convenience of the user can be improved.

Further, the driving of the arm unit 30 may be controlled so that the arm unit 30 performs pivot operation. Here, the pivot operation is an operation of moving the microscope unit 14 so that the optical axis of the microscope unit 14 is oriented to a predetermined point in the space (hereinafter, referred to as a pivot point) at all times. By the pivot operation, the same observation position can be observed from various directions, and therefore more detailed observation of an affected part becomes possible. In the case where the microscope unit 14 is configured such that its focal distance is unadjustable, it is preferable that the pivot operation be performed in a state where the distance between the microscope unit 14 and the pivot point is fixed. In this case, the distance between the microscope unit 14 and the pivot point may be adjusted to the fixed focal distance of the microscope unit 14. Thereby, the microscope unit 14 moves on a hemisphere surface having a radius corresponding to the focal distance, with the pivot point as the center (schematically illustrated in FIG. 1 and FIG. 2), and a clear captured image is obtained even when the observation direction is altered.

On the other hand, in the case where the microscope unit 14 is configured such that its focal distance is adjustable, the pivot operation may be performed in a state where the distance between the microscope unit 14 and the pivot point is variable. In this case, for example, the control device 100 may calculate the distance between the microscope unit 14 and the pivot point on the basis of information concerning the rotation angle of each joint unit detected by the encoder, and may adjust the focal distance of the microscope unit 14 automatically on the basis of the calculation result. Alternatively, in the case where the microscope unit 14 is provided with an AF function, the focal distance may be adjusted automatically by the AF function every time the distance between the microscope unit 14 and the pivot point changes by the pivot operation.

<1-3. Examples of the Use of the Surgical Navigation System>

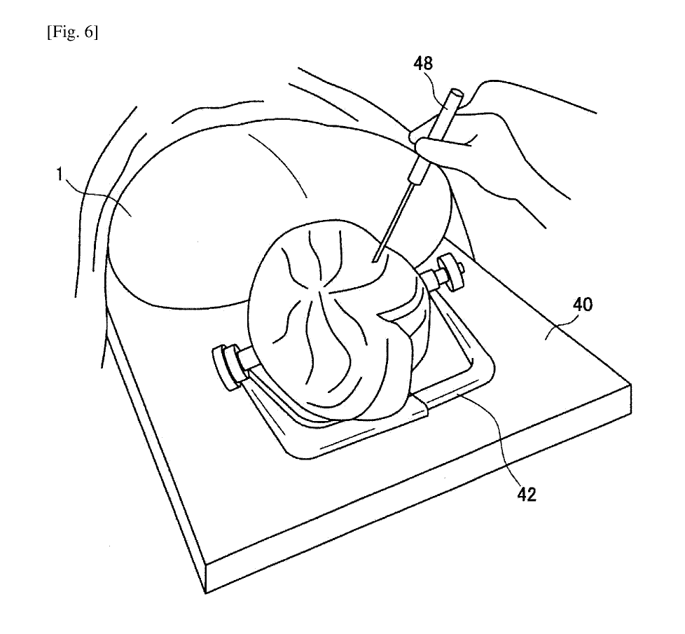

FIG. 5 is a diagram showing an example of the use of the surgical navigation system shown in FIG. 1. In FIG. 5, a situation in which, using the surgical navigation system, an operator 3401 performs an operation on the patient 1 on the bed 40 as a support base that supports the patient 1 is schematically shown. In FIG. 5, the surgical navigation system is simplified for illustration for ease of understanding.

As shown in FIG. 5, during the operation, a surgical field image photographed by the imaging apparatus 10 is displayed with magnification on the display device 54. The display device 54 is installed in a position easily viewable from the operator 3401, and the operator 3401 performs various treatments, such as the excision of an affected part, on a surgical site while observing the condition of the surgical site using a video image shown on the display device 54. The surgical instrument used may be, for example, a surgical instrument equipped with a pair of forceps, a grasper, or the like at its tip, or any of various surgical instruments such as an electric scalpel and an ultrasonic scalpel.

During the operation, an image in which a surgical field image captured by the imaging apparatus 10 is superimposed on a preoperative image or a 3D model is displayed on the display device 54. The operator 3401 performs various treatments, such as the excision of an affected part, in accordance with navigation display displayed on the display device 54 while observing the condition of the surgical site using a video image shown on the display device 54. At this time, on the display device 54, for example, information such as the position of incision, the position of excision, and the position or posture of the tip of a surgical instrument may be displayed.

Hereinabove, an overview of the surgical navigation system to which the technology according to the present disclosure can be applied is described. Some specific embodiments of the technology according to the present disclosure will now be described. In each embodiment described below, an example in which a stereo camera 14A that enables 3D display is used as the microscope unit 14 is described.

### 2. First Embodiment

<2-1. Overview of the Surgical Navigation System>

In a surgical navigation system according to a first embodiment of the present disclosure, the arm unit 30 of the imaging apparatus 10 is fixed to the bed 40 (see FIG. 1). That is, the positional relationship between a fixed portion 32 fixed to the bed 40 of the arm unit 30 and the patient 1 can be kept fixed. Hence, the imaging apparatus 10 according to the embodiment is configured so as to calculate a predetermined position in a three-dimensional coordinate system in which the fixed portion 32 of the arm unit 30 or an arbitrary spatial position having a fixed relative positional relationship with the fixed portion 32 is taken as the origin (reference position) P0. The surgical navigation system according to the embodiment is an example of the system in which neither a reference marker for setting the position of the origin P0 of the three-dimensional coordinates nor a surgical instrument marker for identifying the position or posture of a surgical instrument is used.

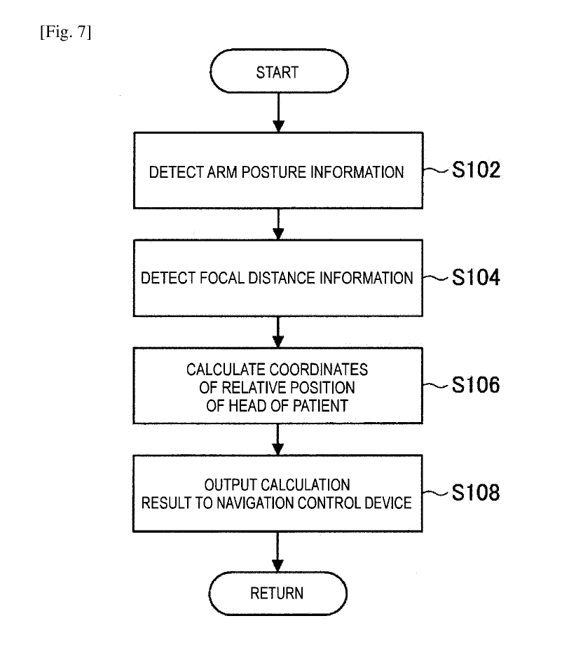

FIG. 6 is an illustration diagram showing a situation of an operation for which the surgical navigation system according to the embodiment can be used. The illustrated example shows a situation of a brain surgery, and the patient 1 is supported on the bed 40 in a state of facing down and the head is fixed by a fixing tool 42. As described above, neither a reference marker for setting the position of the origin P0 of the three-dimensional coordinates nor a surgical instrument marker for indicating the position or posture of a surgical instrument is used.

<2-2. Control Processing>

The control processing executed in the surgical navigation system according to the embodiment will now be described with reference to FIG. 3 and FIG. 4. As the control processing, the processing of grasping a surgical field, registration processing, and the processing of detecting the position of the tip of a surgical instrument are described.

(2-2-1. Processing of Grasping a Surgical Field)

First, an example of the processing of grasping a surgical field imaged by the stereo camera 14A is described. The processing of grasping a surgical field may be a processing for sharing an in-focus position in the captured image obtained by the stereo camera 14A with the navigation apparatus 50. During the operation, since the focus is placed on the surgical site of the patient 1 automatically or by the user's manipulation, the in-focus position can be said to be the position of the surgical site. The in-focus position can be grasped on the basis of the focal distance, the magnification, the angle of view, etc. of the stereo camera 14A.

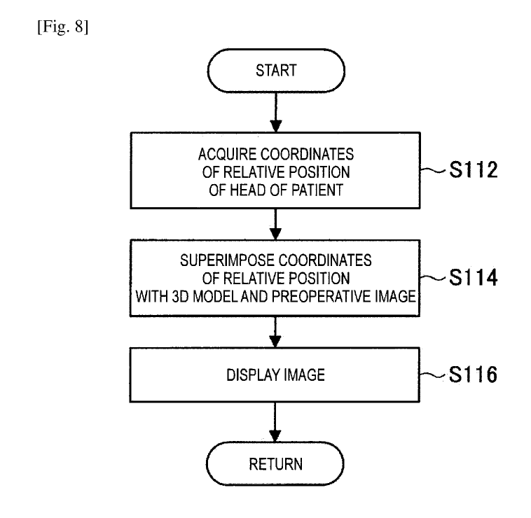

FIG. 7 is a flow chart executed by the control device 100 of the imaging apparatus 10 in the processing of grasping a surgical field. In step S102, in a state where the focus is placed on the head of the patient 1, the arm posture information detection unit 112 detects the posture information of the arm unit 30 on the basis of information concerning the rotation angle of each joint unit detected by the encoder 16 provided in each joint unit of the arm unit 30.

Subsequently, in step S104, the camera information detection unit 114 acquires information outputted from the stereo camera 14A. The information outputted from the stereo camera 14A may include the information of the focal distance, the magnification, the angle of view, etc. of the stereo camera 14A (hereinafter, occasionally referred to as “camera parameters”). The focal distance of the stereo camera 14A may be outputted while being replaced with, for example, the information of the distance in the optical axis direction from the end rotation axis on the stereo camera 14A side in the arm unit 30 to the head of the patient 1. The focal distance, the magnification, the angle of view, etc. of the stereo camera 14A may be altered by the manipulation input of the camera manipulation interface 12, and the set values thereof may be detected by a potentiometer or the like provided in the lens portion of the stereo camera 14A.

Subsequently, in step S106, on the basis of the posture information of the arm unit 30 and the information of the focal distance of the stereo camera 14A, the position calculation unit 116 calculates the relative position of the head of the patient 1 to a predetermined reference position of which the position does not change even when the posture of the arm unit 30 changes. For example, the position calculation unit 116 may calculate the relative three-dimensional coordinates of the head of the patient 1 in a coordinate system (an xyz three-dimensional coordinate system) in which an arbitrary position in the fixed portion 32 of the arm unit 30 fixed to the bed 40 is taken as the origin P0. The origin P0 may be also an arbitrary position having a fixed relative positional relationship with the fixed portion 32 of the arm unit 30.

Subsequently, in step S108, the position calculation unit 116 transmits the calculated relative three-dimensional coordinates of the head of the patient 1 to the navigation control device 60. The position calculation unit 116 performs step S102 to step S108 when at least the posture of the arm unit 30 or any one of the focal distance, the magnification, the angle of view, etc. of the stereo camera 14A is altered. Alternatively, step S102 to step S108 may be performed repeatedly at a predetermined time interval that is set in advance.

FIG. 8 is a flow chart executed by the navigation control device 60 of the navigation apparatus 50 in the processing of grasping a surgical field. In step S112, the navigation control device 60 acquires the relative position of the head of the patient 1 from the control device 100 of the imaging apparatus 10. Subsequently, in step S114, the navigation control device 60 calls up, from the memory device 56, at least one of a 3D model and a preoperative image of the head of the patient 1 of which the relative positional relationship with the origin P0 is found in advance, and superimposes the relative position of the head of the patient 1 transmitted from the position computation unit 110 to produce 3D image information for display. Subsequently, in step S116, the navigation control device 60 outputs the produced 3D image information to the display device 54, and causes the display device 54 to display the image.

The navigation control device 60 may perform step S112 to step S116 repeatedly when the relative position of the head of the patient 1 transmitted from the control device 100 is altered, or at a predetermined time interval that is set in advance. The way of superimposition in the captured image displayed may be designed to be alterable by manipulating the navigation manipulation interface 52.

In order to adjust the surgical field, the user may manipulate the navigation manipulation interface 52 to transmit a control command of the arm unit 30 to the arm posture control unit 120 via the navigation control device 60. Alternatively, a design in which the navigation control device 60 itself can transmit a control command of the arm unit 30 to the arm posture control unit 120 on the basis of a predetermined arithmetic processing is possible. The arm posture control unit 120 resolves the control command of the arm unit 30 into the operation of each joint unit, and outputs the resolved control command to the motor 18 of each joint unit as the instruction value of the rotation angle and/or the amount of movement. The manipulation of the arm unit 30 may also be performed directly by the manipulation of the arm manipulation interface 20 by the user without using the navigation control device 60.

(2-2-2. Registration Processing)

Next, an example of the processing of registration between the head of the patient 1 in the captured image and a preoperative image or reference points present in a 3D model, a preoperative image, or the like is described. In the registration processing, the head of the patient 1 in the captured image acquired by the stereo camera 14A, a preoperative image or a 3D model produced from an MRI image or the like photographed prior to the operation, and reference points are registered.

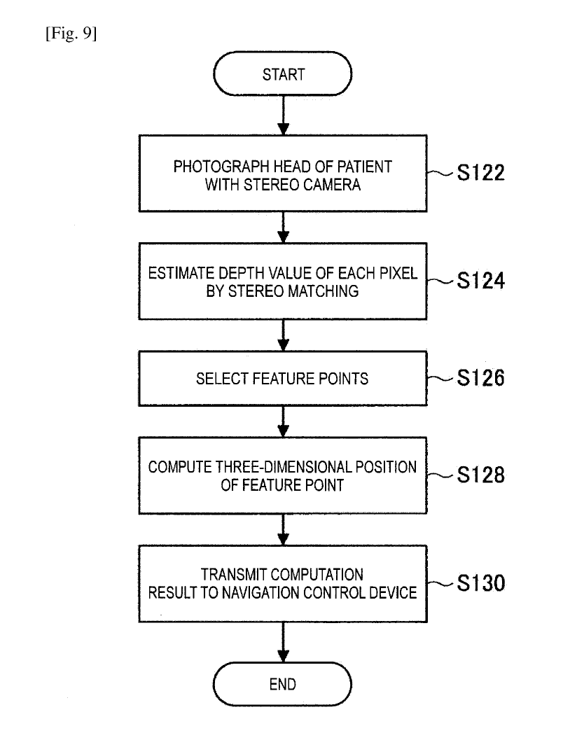

FIG. 9 shows a flow chart of registration processing. First, in step S122, the camera information detection unit 114 of the position computation unit 110 of the control device 100 acquires 3D image information outputted from the stereo camera 14A. Here, the head of the patient 1 is photographed by the stereo camera 14A. Subsequently, in step S124, the position calculation unit 116 estimates the depth value of each pixel by the stereo matching method on the basis of captured images produced on the basis of the 3D image information acquired by the stereo camera 14A and the camera parameters. The depth value may be estimated by utilizing known technology.

Subsequently, in step S126, the position calculation unit 116 computes the shape change (undulation) around the obtained depth value, and extracts an arbitrary number of feature points with a large undulation. The number of feature points may be three or more, for example. Subsequently, in step S128, the position calculation unit 116 calculates the relative three-dimensional coordinates of the extracted feature point. At this time, the detected value of the encoder 16 of each joint unit detected by the arm posture information detection unit 112 and the camera parameters of the stereo camera 14A are utilized to obtain the relative three-dimensional coordinates, with the fixed portion 32 of the arm unit 30 or the like as the reference position.

Subsequently, in step S130, the position calculation unit 116 transmits the 3D image information captured by the stereo camera 14A and the information of the relative three-dimensional coordinates of the feature point to the navigation control device 60. Thereby, in the navigation control device 60, the comparison and matching between the position of the feature point and the position of the corresponding reference point in the preoperative image or the 3D model can be performed, and the comparison result may be displayed on the display device 54. Viewing the displayed comparison result, the user adjusts the posture of the arm unit 30 so that the head of the patient 1 in the captured image and the preoperative image or the 3D model are registered.

In the surgical navigation system according to the embodiment, the arm unit 30 equipped with the stereo camera 14A is fixed to the bed 40, and the positional relationship with the head of the patient 1 can be kept fixed; thus, once one registration processing is performed, it is not necessary to perform registration again during the operation. Furthermore, the surgical navigation system according to the embodiment finds the relative position with respect to, as the reference position, the fixed portion 32 of the arm unit 30 having a fixed positional relationship with the head of the patient 1; therefore, it is not necessary to find the absolute position of the head of the patient 1 in the three-dimensional space and a reference marker is not necessary.

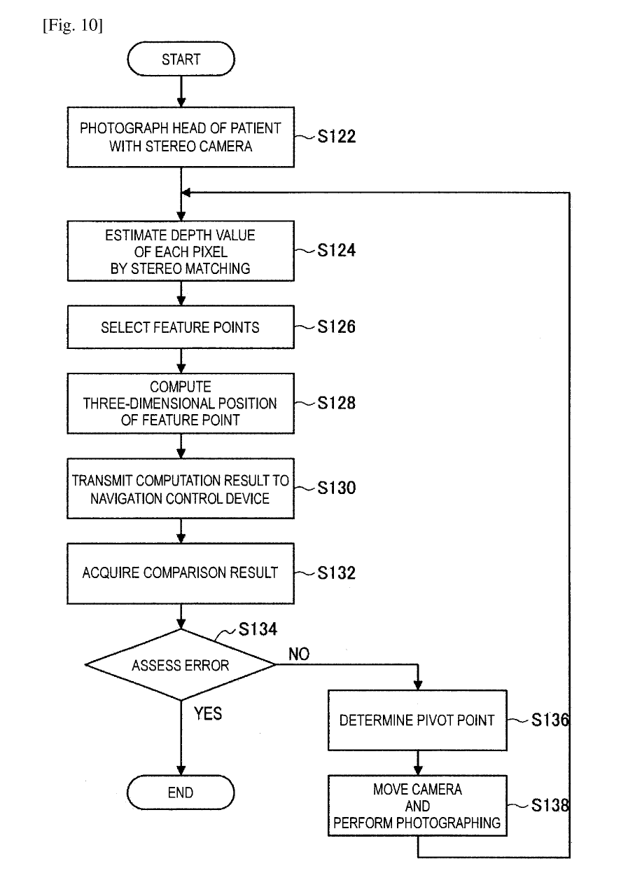

The posture of the arm unit 30 may be adjusted also by automatic correction control by the arm posture control unit 120 without using the user's manipulation. FIG. 10 is a flow chart of the automatic registration processing performed by the arm posture control unit 120. The position computation unit 110 of the control device 100 performs step S122 to step S130 in accordance with the flow chart shown in FIG. 9. In step S132, the arm posture control unit 120 of the control device 100 acquires, from the navigation control device 60, the result of comparison between the position of the feature point and the position of the corresponding reference point in the preoperative image or the 3D model.

Subsequently, in step S134, the arm posture control unit 120 assesses the error between the position of the feature point and the position of the reference point in the preoperative image or the 3D model. For example, the arm posture control unit 120 may determine whether or not the distance between the relative three-dimensional coordinate position of the feature point and the relative three-dimensional coordinate position of the reference point in the preoperative image or the 3D model falls within less than a previously set threshold. In the case where the result of assessment of the error shows that there is a large discrepancy between the position of the feature point and the position of the corresponding reference point in the preoperative image or the 3D model (S134: No), the arm posture control unit 120 goes to step S136 and determines the pivot point at the time of moving the position of the stereo camera 14A. For example, the arm posture control unit 120 may calculate the position of a virtual center of the head of the patient 1 that is stereoscopically reconstructed, and may take the position of the virtual center as the pivot point.

Subsequently, in step S138, on the basis of the amount of discrepancy and the direction of discrepancy between the position of the feature point and the position of the reference point, the arm posture control unit 120 controls the motor 18 of each joint unit of the arm unit 30 to put the stereo camera 14A into pivot operation with the pivot point as the center, and then performs photographing with the stereo camera 14A. After that, the procedure returns to step S124, and the processing of step S124 to step S134 described above is performed repeatedly. Then, in the case where the result of assessment of the error in step S134 shows that there is not a large discrepancy between the position of the feature point and the position of the corresponding reference point in the preoperative image or the 3D model (S134: Yes), the arm posture control unit 120 finishes the registration processing.

When automatic registration processing by the arm posture control unit 120 is possible, the position of the stereo camera 14A can be moved to an appropriate position, and thus the head of the patient 1 in the captured image and the preoperative image or the 3D model can be registered easily, without using adjustment by the user. Also in the case where automatic registration processing is performed, in the surgical navigation system according to the embodiment, after one registration processing is performed, registration is not performed again during the operation.

(2-2-3. Processing of Detecting the Position of a Surgical Instrument)

Next, an example of the processing of detecting the position of the tip of a surgical instrument is described. During the operation, for example as shown in FIG. 6, there is a case where a probe 48 that is a surgical instrument dedicated to position detection is put on the surface of the brain in an attempt to find the positional relationship between the position of the probe 48 and a reference point on a preoperative image or on a 3D model of the surgical site. Specifically, there may occur a situation in which it is desired to find the position of the tip of a surgical instrument accurately when neither a microscope nor a video microscope is used as a camera, alternatively when a microscope or the like is used and yet it is desired to find a more accurate position pin-pointedly, or when the tip of the surgical instrument is buried in the brain parenchyma.

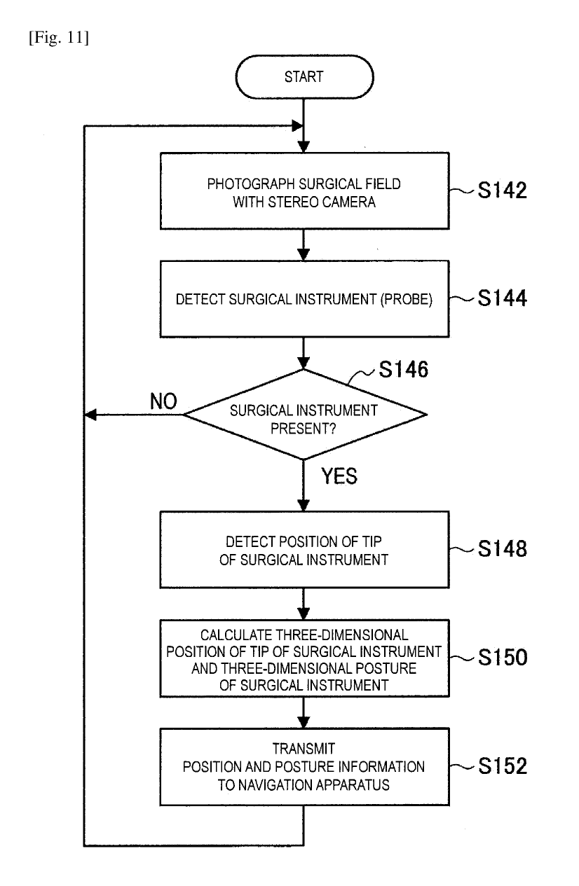

FIG. 11 is a flow chart executed by the control device 100 of the imaging apparatus 10 in the processing of detecting the position of the tip of the probe 48. The flow chart may be basically executed after the registration processing shown in FIG. 9 and FIG. 10. That is, the processing of detecting the position of the tip of the probe 48 may be executed in a state where the relative positions between the head of the patient 1 and the stereo camera 14A are determined.

First, in step S142, the camera information detection unit 114 of the position computation unit 110 of the control device 100 acquires 3D image information outputted from the stereo camera 14A. Here, the head of the patient 1 is photographed by the stereo camera 14A. Subsequently, in step S144, the position calculation unit 116 performs image processing on a captured image produced on the basis of the 3D image information acquired by the stereo camera 14A, and thereby attempts to detect the probe 48. For example, the position calculation unit 116 attempts to detect the probe 48 in the captured image by the processing of matching with the shape of the grasping portion of the probe 48, the shape of the connection portion between the grasping portion and the tip portion of the probe 48, or the like stored in advance.

Subsequently, in step S146, the position calculation unit 116 determines whether the probe 48 is detected in the captured image or not. In the case where the probe 48 is not detected in the captured image (S146: No), the procedure returns to step S142, and step S142 to step S146 are repeated until the probe 48 is detected. On the other hand, in the case where in step S146 the probe 48 is detected in the captured image (S146: Yes), the position calculation unit 116 calculates the position of the tip of the probe 48 in step S148. For example, the position calculation unit 116 may detect the position of the tip of the probe 48 on the basis of the information of the shape and length of the probe 48 stored in advance.

Further, in step S150, the position calculation unit 116 calculates the relative three-dimensional coordinates of the tip of the probe 48 and the posture of the probe 48 in the three-dimensional coordinate space. The posture of the probe 48 may be calculated by, for example, image processing. Subsequently, in step S152, the position calculation unit 116 transmits the calculated relative position of the tip of the probe 48 and the calculated posture information of the probe 48 to the navigation control device 60. After that, the procedure returns to step S142, and step S142 to step S152 are repeated.

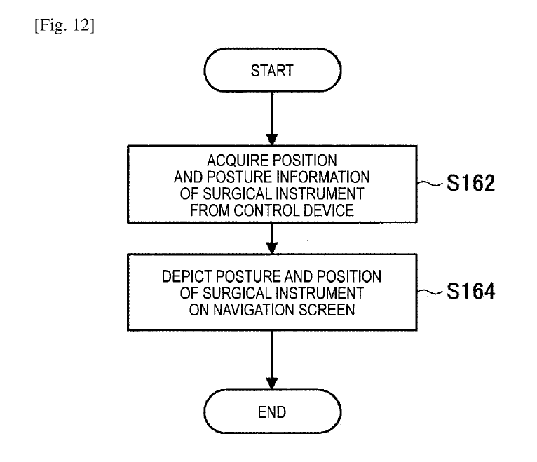

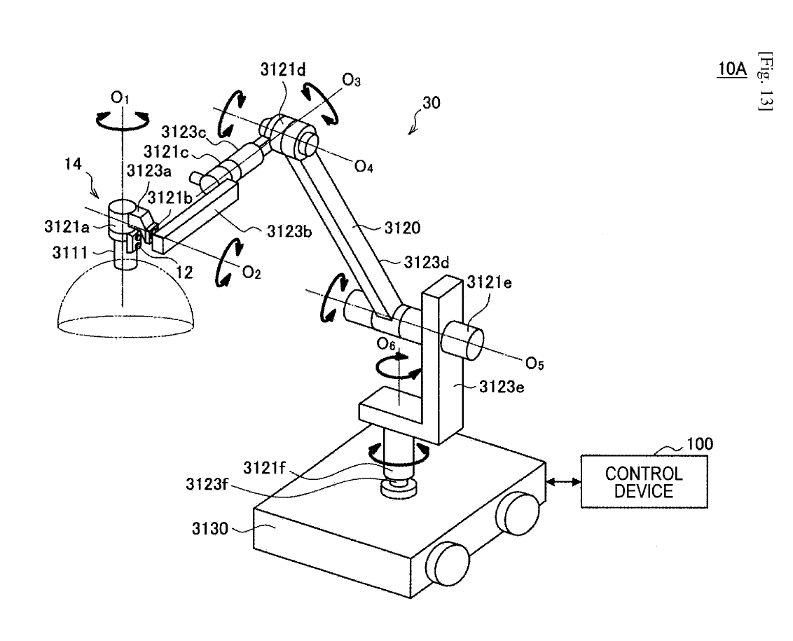

FIG. 12 is a flow chart executed by the navigation control device 60 of the navigation apparatus 50 in the processing of detecting the position of the probe 48. In step S162, the navigation control device 60 acquires, from the control device 100 of the imaging apparatus 10, the relative position information of the tip of the probe 48 and the posture information of the probe 48. Subsequently, in step S164, the navigation control device 60 depicts the probe 48 on the image information of the head of the patient 1 for which registration has been completed, and causes the display device 54 to display the image of the probe 48 in real time. Thereby, the operator can move the tip of the probe 48 to a desired position while viewing navigation display displayed on the display device 54.

<2-3. Conclusions>

Thus, by the imaging apparatus 10 and the surgical navigation system according to the embodiment, a predetermined position can be calculated on the basis of the posture information of the arm unit 30 equipped with the stereo camera 14A and the information outputted from the stereo camera 14A. Therefore, it is not necessary to add a sensor such as an optical sensor or a magnetic sensor separately from the imaging apparatus 10. Thus, the setting of a sensor is not necessary, and false detection and an undetectable state due to a disturbance such as an optical shield, a magnetic shield, or noise can be eliminated. Furthermore, the number of equipment parts in the surgical navigation system can be reduced, and the cost can be reduced.

Furthermore, by the imaging apparatus 10 according to the embodiment, the relative three-dimensional coordinates of a surgical site imaged by the stereo camera 14A can be calculated on the basis of the posture information of the arm unit 30 and the camera parameters such as the focal distance of the stereo camera 14A. Therefore, the relative position of the surgical site can be detected and utilized for navigation control, without using an additional sensor.

Furthermore, by the imaging apparatus 10 according to the embodiment, the relative three-dimensional coordinates of the feature point of the surgical site can be calculated on the basis of the posture information of the arm unit 30, and the 3D image information and the camera parameters outputted from the stereo camera 14A. Therefore, the registration of the surgical site can be easily performed in the navigation apparatus 50 without using an additional sensor. In addition, when the result of matching between the captured image and a preoperative image is fed back to the posture control of the arm unit 30, automatic registration of the surgical site becomes possible, and registration working is simplified.

Moreover, by the imaging apparatus 10 according to the embodiment, the position and posture of a surgical instrument or the tip of a surgical instrument can be calculated on the basis of the posture information of the arm unit 30, and the 3D image information and the camera parameters outputted from the stereo camera 14A. Therefore, without using an additional sensor, the position and posture of the surgical instrument or the tip of the surgical instrument can be accurately detected in the navigation apparatus 50, and the surgical instrument can be superimposed and displayed on the display device 54 accurately in real time. Thereby, even when the tip of the surgical instrument has entered the interior of the body, the operator can move the tip of the surgical instrument to a desired position.

### 3. Second Embodiment

<3-1. Overview of the Surgical Navigation System>

In a surgical navigation system according to a second embodiment of the present disclosure, the arm unit 30 of an imaging apparatus 10A is mounted on a movable cart. That is, the arm unit 30 is not fixed to the bed 40, and any position of the arm unit 30 can change with respect to the patient 1; hence, it is necessary to perform the processing of setting the origin of the three-dimensional coordinates. Thus, in the surgical navigation system according to the embodiment, a reference marker 134 is used to set the origin (reference position) P0 of the three-dimensional coordinates.

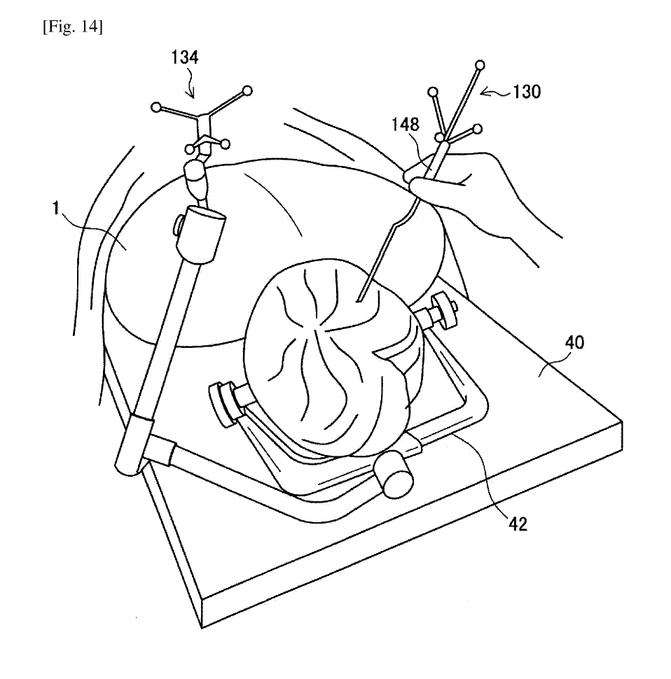

FIG. 13 is an illustration diagram showing an example of the configuration of the imaging apparatus 10A used in the surgical navigation system according to the embodiment. The imaging apparatus 10A may be configured in a similar manner to the imaging apparatus 10 shown in FIG. 2 except that the arm unit 30 is mounted on a movable cart 3130. The imaging apparatus 10A may be placed in an arbitrary position on a side of the bed 40 by the user.

FIG. 14 is an illustration diagram showing a situation of an operation for which the surgical navigation system according to the embodiment can be used. The illustrated example shows a situation of a brain surgery, and the patient 1 is supported on the bed 40 in a state of facing down and the head is fixed by the fixing tool 42. A reference marker 134 is connected to the fixing tool 42 via a connecting jig. That is, the positional relationship between the reference marker 134 and the patient 1 can be kept fixed. Thus, the imaging apparatus 10A according to the embodiment is configured so as to detect a predetermined position in a three-dimensional coordinate system in which a predetermined position specified on the basis of the three-dimensional position of the reference marker 134 is taken as the origin P0. In the surgical navigation system according to the embodiment, a surgical instrument 148 includes a surgical instrument marker 130, and the surgical instrument marker 130 is utilized to detect the position and posture of the surgical instrument 148.

The reference marker 134 and the surgical instrument marker 130 may be an optical marker including four marker units serving as marks for detecting the position or posture. For example, a configuration in which a marker unit that diffusely reflects light of a wavelength in the infrared region emitted from a light source is used and the position and posture of the marker are detected on the basis of 3D image information acquired by a stereo camera 14A having sensitivity at the wavelength in the infrared region is possible. Alternatively, a configuration in which a marker unit with a distinctive color such as red is used and the position and posture of the marker are detected on the basis of 3D image information acquired by a stereo camera 14A is possible. Since the positional relationships among the four marker units in the captured image vary with the position and posture of the marker, the position calculation unit 116 can identify the position and posture of the marker by detecting the positional relationships among the four marker units.

<3-2. Position Detection Processing>

The control processing executed in the surgical navigation system according to the embodiment will now be described with reference to FIG. 3 and FIG. 4.

(3-2-1. Processing of Grasping a Surgical Field)

First, the processing of grasping a surgical field executed by the control device 100 of the imaging apparatus 10A according to the embodiment is described. The processing of grasping a surgical field is basically executed in accordance with the flow chart shown in FIG. 7. However, in the imaging apparatus 10A according to the embodiment, a predetermined position specified on the basis of the reference marker 134 is taken as the origin P0 of the three-dimensional coordinate system. Therefore, in step S106 of FIG. 7, on the basis of the posture information of the arm unit 30 and the information of the focal distance of the stereo camera 14A, the position calculation unit 116 calculates the relative three-dimensional coordinates of the head of the patient 1 of which the origin P0 is the predetermined position specified on the basis of the reference marker 134. The origin P0 may be set in advance as, for example, the position of the three-dimensional coordinates of the reference marker 134 calculated on the basis of the posture information of the arm unit 30 and the camera parameters outputted from the stereo camera 14A.

The position of the reference marker 134 serving as the origin P0 may be the position of any one of the four marker units of the reference marker 134, or may be an arbitrary position that is other than the marker unit and has a fixed relative position to the reference marker 134. The three-dimensional coordinates with respect to the arbitrary origin P0 may be defined by the posture of the reference marker 134. That is, the position calculation unit 116 may specify the three axes of x, y, and z on the basis of the posture of the identified reference marker 134. Thereby, the position calculation unit 116 can find the relative three-dimensional coordinates of the head of the patient 1 to the origin P0.

In the surgical navigation system according to the embodiment, the processing of grasping a surgical field can be executed in a similar manner to the case of the processing of grasping a surgical field by the surgical navigation system according to the first embodiment except that the three-dimensional position is calculated as the relative three-dimensional coordinates to the origin P0 specified by the reference marker 134.

(3-2-2. Registration Processing)

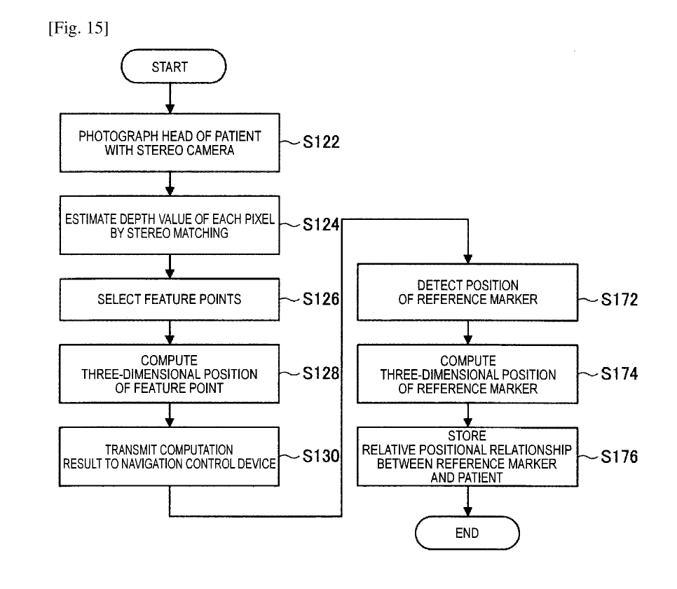

Next, an example of the processing of registration between the head of the patient 1 in the captured image and a preoperative image or reference points present in a 3D model, a preoperative image, or the like is described. FIG. 15 shows a flow chart of the registration processing.

Also in the control device 100 of the imaging apparatus 10A according to the embodiment, first, step S122 to step S130 are performed in accordance with a similar procedure to the flow chart shown in FIG. 9. Thereby, the comparison and matching between the position of the feature point and the position of the corresponding reference point in the preoperative image or the 3D model are performed in the navigation control device 60, and the comparison result is displayed on the display device 54. Viewing the displayed comparison result, the user adjusts the posture of the arm unit 30 so that the head of the patient 1 in the captured image and the preoperative image or the 3D model are registered.

When the registration between the head of the patient 1 and the preoperative image or the 3D model is completed, in step S172, the camera information detection unit 114 acquires 3D image information outputted from the stereo camera 14A. Here, the reference marker 134 is photographed by the stereo camera 14A. The position of the stereo camera 14A may move as long as the movable cart 3130 equipped with the arm unit 30 does not move. Subsequently, in step S174, the position calculation unit 116 calculates the three-dimensional coordinates of the reference marker 134 on the basis of the posture information of the arm unit 30 and the camera parameters outputted from the stereo camera 14A, and sets a predetermined position specified by the reference marker 134 as the origin P0.

Subsequently, in step S176, the position calculation unit 116 calculates and stores the relative three-dimensional coordinates of the head of the patient 1 to the origin P0 specified by the reference marker 134. The information of the relative three-dimensional coordinates of the head of the patient 1 may be also transmitted to and stored in the navigation apparatus 50.

In the surgical navigation system according to the embodiment, since the stereo camera 14A is mounted on the movable cart 3130 to be made movable, when the position of the movable cart 3130 has changed, registration processing is executed again. In other words, as long as the relative positional relationship between the head of the patient 1 and the reference marker 134 does not change and the position of the movable cart 3130 does not change either, once one registration processing is performed, it is not necessary to perform registration again during the operation. Also in the surgical navigation system according to the embodiment, automatic registration processing may be performed in accordance with the flow chart shown in FIG. 10.

(3-2-3. Processing of Detecting the Position of a Surgical Instrument)

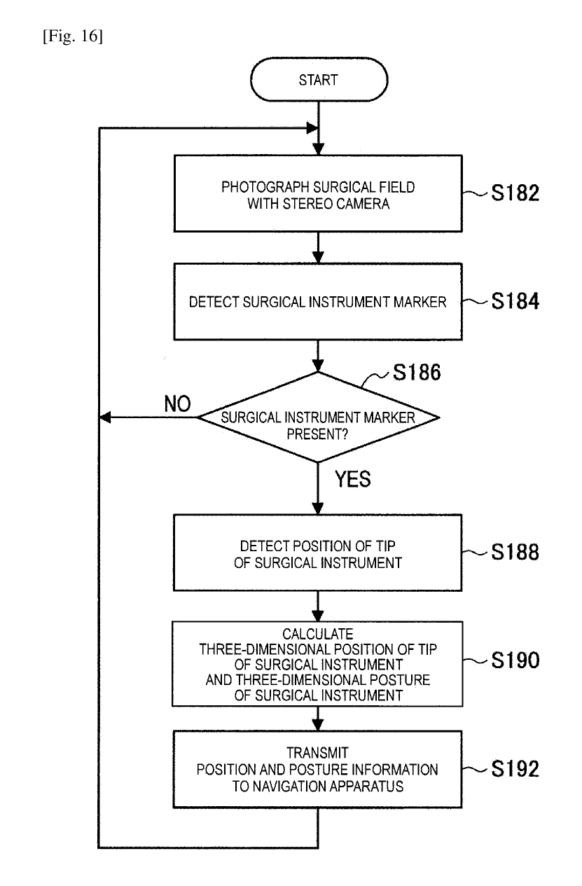

Next, an example of the processing of detecting the position of the tip of a surgical instrument is described. FIG. 16 is a flow chart executed by the control device 100 of the imaging apparatus 10A in the processing of detecting the position of the tip of a surgical instrument dedicated to position detection (a probe) 148. The flow chart may be basically executed after the registration processing shown in FIG. 15. That is, the processing of detecting the position of the tip of the probe 148 may be executed in a state where the origin P0 of the three-dimensional coordinates and the relative positions between the head of the patient 1 and the stereo camera 14A are determined.

First, in step S182, the camera information detection unit 114 of the position computation unit 110 of the control device 100 acquires 3D image information outputted from the stereo camera 14A. Here, the head of the patient 1 is photographed by the stereo camera 14A. Subsequently, in step S184, it is attempted to detect the surgical instrument marker 130 from a captured image produced on the basis of the 3D image information acquired by the stereo camera 14A. Subsequently, in step S186, the position calculation unit 116 determines whether the surgical instrument marker 130 is detected in the captured image or not. In the case where the surgical instrument marker 130 is not detected in the captured image (S186: No), the procedure returns to step S182, and step S182 to step S186 are repeated until the surgical instrument marker 130 is detected.

On the other hand, in the case where in step S186 the surgical instrument marker 130 is detected in the captured image (S186: Yes), the position calculation unit 116 detects the position of the tip of the probe 148 in step S188. For example, the position calculation unit 116 may detect the position of the tip of the probe 148 on the basis of the information of the shape and length of the probe 148 stored in advance. Further, in step S190, the position calculation unit 116 calculates the relative three-dimensional coordinates of the tip of the probe 148 to the origin P0 specified by the reference marker 134 and the posture of the probe 148 in the three-dimensional space. Subsequently, in step S192, the position calculation unit 116 transmits the calculated relative position of the tip of the probe 148 and the calculated posture information of the probe 148 to the navigation control device 60. After that, the procedure returns to step S182, and step S182 to step S192 are repeated.

In accordance with the flow chart shown in FIG. 12, the navigation control device 60 acquires, from the control device 100 of the imaging apparatus 10, the relative position of the tip of the probe 148 and the posture information of the probe 148, depicts the probe 148 on the image information of the head of the patient 1, and causes the display device 54 to display the image of the probe 148 in real time. Thereby, even when the tip of the probe 148 has entered the interior of the body, the operator can move the tip of the surgical instrument to a desired position while viewing navigation display displayed on the display device 54.

(3-2-4. Positional Shift Examination Processing)

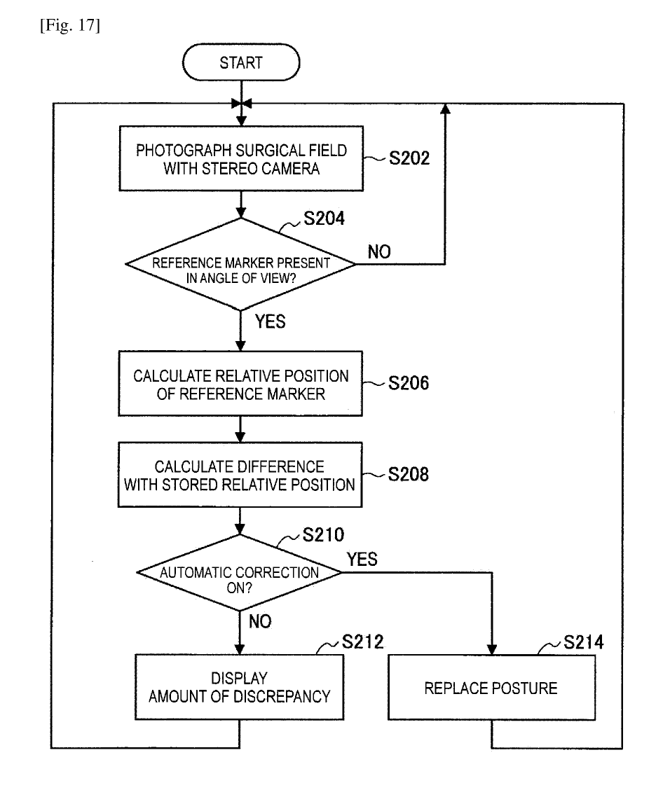

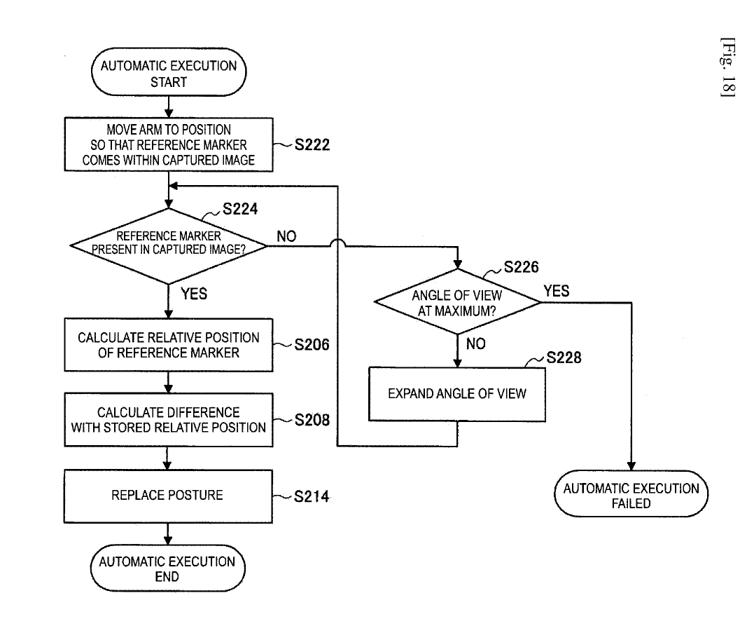

Next, the processing of examining the positional shift of the arm unit 30 is described. In the surgical navigation system according to the embodiment, since the reference marker 134 is used, the positional shift of the arm unit 30 due to a movement of the movable cart 3130 or the like can be examined. FIG. 17 is a flow chart showing the processing of examining the positional shift of the arm unit 30. The flow chart is a procedure in which, when the reference marker 134 appears on the screen during an operation or working, the image information of the reference marker 134 is utilized to examine the positional shift of the arm unit 30, and is basically executed after the registration processing shown in FIG. 15. That is, the processing of positional shift examination may be executed in a state where the origin P0 of the three-dimensional coordinates specified on the basis of the reference marker 134 and the relative positions between the head of the patient 1 and the stereo camera 14A are determined.

First, in step S202, the camera information detection unit 114 of the position computation unit 110 of the control device 100 acquires 3D image information outputted from the stereo camera 14A. Subsequently, in step S204, the position calculation unit 116 determines whether the reference marker 134 is present in a captured image produced on the basis of the 3D image information acquired by the stereo camera 14A or not. In the case where the reference marker 134 is not present in the captured image (S204: No), the positional shift of the arm unit 30 cannot be examined and thus the procedure returns to step S202.

In the case where the reference marker 134 is present in the captured image (S204: Yes), in step S206 the position calculation unit 116 calculates the three-dimensional coordinates of the reference marker 134 with respect to the origin P0. That is, in step S206, the relative position of the reference marker 134 to the origin P0 is calculated. Subsequently, in step S208, the position calculation unit 116 calculates the difference between the relative position of the reference marker 134 calculated in step S206 and the relative position of the reference marker 134 at the time point when the current origin P0 is set. For example, the difference between the components in each axis direction of the three-dimensional coordinates corresponding to the relative positions is found. When a positional shift of the arm unit 30 has not occurred, the difference between the relative positions mentioned above is zero.

Subsequently, in step S210, the position calculation unit 116 determines whether the automatic correction mode is ON or not. In the case where the automatic correction mode is OFF (S210: No), in step S212, the position calculation unit 116 transmits the amount of discrepancy of the relative position of the reference marker 134 found in step S208 to the navigation control device 60, and causes the display device 54 to display the amount of discrepancy. Thereby, the user can find the presence or absence of positional shift of the arm unit 30; and when the user considers the amount of discrepancy to be large, the user oneself may move the arm unit 30 while setting the automatic correction mode to ON, and can thereby correct the positional shift of the arm unit 30 clearly.

On the other hand, in the case where the automatic correction mode is ON (S210: Yes), in step S214 the position calculation unit 116 performs the replacement of the posture information of the arm unit 30. The replacement of the posture information of the arm unit 30 may be performed by, for example, correcting the posture information of the arm unit 30 corresponding to the relative position of the reference marker 134 calculated this time. Thereby, after the replacement of the posture information of the arm unit 30 is performed, the position calculation unit 116 calculates the posture information of the arm unit 30 using the difference with the posture information of the arm unit 30 after the replacement, and utilizes the calculation result for various computations such as position detection.

By executing positional shift examination processing in the above way, the accuracy of the posture information of the arm unit 30 can be assessed any time by capturing the reference marker 134 in the captured image. Furthermore, for example, when the movable cart 3130 equipped with the arm unit 30 has moved, the position information of the reference marker 134 captured may be utilized to detect the shift of the posture of the arm unit 30, and the posture information of the arm unit 30 may be replaced; thereby, accurate position information can be calculated at all times.

Although in the example of the flow chart shown in FIG. 17 the positional shift of the arm unit 30 is measured by comparing the relative positions of the reference marker 134, the positional shift of the arm unit 30 may be measured also by using the posture information of the arm unit 30 in a state where the reference marker 134 is captured.

Further, the control device 100 may operate so as to capture the reference marker 134 in the captured image at an appropriate timing, and may execute the examination of positional shift and the automatic correction of the posture information of the arm unit 30. FIG. 18 shows a flow chart of recalibration processing. First, in step S222, in order to execute recalibration, the position calculation unit 116 sends a command to the arm posture control unit 120 to cause the arm posture control unit 120 to change the posture of the arm unit 30 so that the reference marker 134 comes within the captured image of the stereo camera 14A. At this time, the posture control of the arm unit 30 may be performed by the user's manipulation, or automatic posture control of the arm unit 30 may be performed by the control device 100 itself so that the reference marker 134 is detected in the captured image of the stereo camera 14A, on the basis of the currently stored relationship between the position of the head of the patient 1 and the position of the reference marker 134.

Subsequently, in step S224, the position calculation unit 116 determines whether the reference marker 134 is present in the captured image acquired by the stereo camera 14A or not. In the case where the reference marker 134 is present in the captured image (S224: Yes), the position calculation unit 116 performs the replacement of the posture information of the arm unit 30 in accordance with the procedure of step S206, step S208, and step S214 in the flow chart of FIG. 17, and subsequently calculates the posture of the arm unit 30 using the difference with the posture information of the arm unit 30 at this time.

On the other hand, in the case where in step S224 the reference marker 134 is not present in the captured image (S224: No), the procedure goes to step S226, and the position calculation unit 116 determines whether the angle of view of the stereo camera 14A is at the maximum or not. In the case where the angle of view is already at the maximum (S226: Yes), the reference marker 134 cannot be captured by the stereo camera 14A and calibration cannot be automatically executed; hence, the processing is finished. On the other hand, in the case where the angle of view is not at the maximum (S226: No), in step S228 the position calculation unit 116 expands the angle of view of the stereo camera 14A to expand the imaging range; and then the procedure returns to step S224, and step S224 and the subsequent steps are repeated.

Thereby, in the case where the arm unit 30 is not fixed to the bed 40, when the movable cart 3130 equipped with the arm unit 30 has moved, recalibration can be completed automatically when the reference marker 134 is captured in the captured image successfully. When performing calibration, it is also possible to change the posture of the arm unit 30 to move the position of the stereo camera 14A back, instead of or in combination with the expansion of the angle of view of the stereo camera 14A.

<3-3. Conclusions>

Thus, by the imaging apparatus 10A and the surgical navigation system according to the embodiment, a predetermined position can be calculated on the basis of the posture information of the arm unit 30 equipped with the stereo camera 14A and the information outputted from the stereo camera 14A. Therefore, a similar effect to the imaging apparatus 10 according to the first embodiment can be obtained. Also in the imaging apparatus 10A according to the embodiment, the relative three-dimensional coordinates of the surgical site, the relative three-dimensional coordinates of the feature point of the surgical site, and the relative three-dimensional coordinates of the position of a surgical instrument or the tip of a surgical instrument can be detected on the basis of the posture information of the arm unit 30 and the information acquired from the stereo camera 14A. Therefore, the control of the processing of grasping a surgical field, registration processing, the processing of detecting the position of the tip of a surgical instrument, etc. can be performed simply and accurately.

Furthermore, the imaging apparatus 10A and the surgical navigation system according to the embodiment are configured so as to perform position detection processing using the reference marker 134 and the surgical instrument marker 130, and can therefore, after the completion of registration processing, execute the processing of examining the positional shift of the arm unit 30 due to a movement of the movable cart 3130 or the like and automatic calibration processing. Therefore, even when a positional shift of the arm unit 30 has occurred, the reliability of various position detection processings can be maintained.

It should be understood by those skilled in the art that various modifications, combinations, sub-combinations and alterations may occur depending on design requirements and other factors insofar as they are within the scope of the appended claims or the equivalents thereof.

For example, although in each embodiment described above the arm unit 30 includes the microscope unit 14 as a camera, the technology of the present disclosure is not limited to such an example. For example, the arm unit 30 may include an eyepiece-equipped microscope and a camera that records a magnified image obtained via the eyepiece-equipped microscope or even a surgical exoscope.

Furthermore, although in each embodiment described above the information of the value of the depth to a predetermined object part is acquired using a stereo camera as the microscope unit 14, the technology of the present disclosure is not limited to such an example. For example, the information of the depth value may be acquired using a distance sensor together with a monocular camera.

Furthermore, although in the first embodiment the detection of a surgical instrument in the captured image is performed by image processing and in the second embodiment the detection of a surgical instrument in the captured image is performed by the detection of the surgical instrument marker, the method for detecting a surgical instrument in each embodiment may be the opposite. That is, although the first embodiment and the second embodiment are different in the way of the setting of the origin P0 of the three-dimensional coordinates, the method for detecting a surgical instrument is not limited to the examples mentioned above.

Furthermore, although in the embodiments described above the control device 100 of the imaging apparatus includes the position computation unit 110 and the arm posture control unit 120, the technology of the present disclosure is not limited to such an example. In the control device 100 according to an embodiment of the present disclosure, it is sufficient that the information of a predetermined position be able to be calculated on the basis of the posture information of the arm unit 30 and the information outputted from the stereo camera 14A, and the arm posture control unit 120 may not be provided. In this case, the posture control of the arm unit 30 may be performed by some other control device having the function of the arm posture control unit 120.

Moreover, the system configurations and the flow charts described in the embodiments described above are only examples, and the technology of the present disclosure is not limited to such examples. Part of the steps in a flow chart executed by the control device 100 of the imaging apparatus may be executed on the navigation control device side. For example, in the automatic registration processing shown in FIG. 10, step S132 to step S136 of the arm unit 30 may be performed by the navigation control device 60, and the computation result may be transmitted to the control device 100.

The computer program for achieving each function of the imaging apparatus and the surgical navigation system may be installed in any of the control devices and the like. A recording medium readable on a computer in which such a computer program is stored can be provided. The recording medium is, for example, a magnetic disk, an optical disk, a magneto-optical disk, a flash memory, or the like. The computer program mentioned above may also be distributed via a network without using a recording medium, for example.

Further, the effects described in this specification are merely illustrative or exemplified effects, and are not limitative. That is, with or in the place of the above effects, the technology according to the present disclosure may achieve other effects that are clear to those skilled in the art based on the description of this specification.

(1)

A surgical information processing apparatus, including:

circuitry configured to

obtain position information of a surgical imaging device, the position information indicating displacement of the surgical imaging device from a predetermined position,

in a registration mode, obtain first image information from the surgical imaging device regarding a position of a surgical component,

determine the position of the surgical component based on the first image information and the position information,

in an imaging mode, obtain second image information from the surgical imaging device of the surgical component based on the determined position.

(2)

The surgical information processing apparatus according to (1), wherein the position determination is further performed by determining a position of the surgical imaging device with respect to the predetermined position based on the position information and by determining a distance between the surgical component and the surgical imaging device.

(3)

The surgical information processing apparatus according to (1) to (2), wherein the surgical component is one of a surgical site and a surgical instrument.

(4)

The surgical information processing apparatus according to (1) to (3), wherein the circuitry activates the registration mode or the imaging mode based on the position information.

(5)

The surgical information processing apparatus according to (1) to (4), wherein the first image information is obtained in the registration mode at a different perspective that the second image information that is obtained in the imaging mode.

(6)

The surgical information processing apparatus according to (1) to (5), wherein the position determination is further performed by setting the position of the surgical imaging device as a reference point.

(7)

The surgical information processing apparatus according to (1) to (6), wherein the position information of the surgical imaging device is based on arm position information from a supporting arm having attached thereto the surgical imaging device, and

wherein the arm position information includes information of movement of at least one joint in the supporting arm.

(8)

The surgical information processing apparatus according to (7), wherein the information of movement of at least one joint in the supporting arm includes an amount of rotation of each joint.

(9)

The surgical information processing apparatus according to (1) to (8), wherein the position determination is further performed by processing images of the surgical component obtained by the surgical imaging device as the first image information.

(10)

The surgical information processing apparatus according to (9), wherein the processing of the images of the surgical component obtained by the surgical imaging device is based on the focus points of the images.

(11)

The surgical information processing apparatus according to (1) to (10), wherein the position of the surgical component being a reference point for image registration between a previously obtained medical image and images obtained by the surgical imaging device as the second image information.

(12)

The surgical information processing apparatus according to (1) to (11), wherein the position of the surgical component being a reference point for superimposing at least one pre-operative image on images obtained by the surgical imaging device as the second image information.

(13)

A surgical information processing method implemented using circuitry, including: obtaining first position information of a surgical imaging device, the first position information indicating displacement of the surgical imaging device from a predetermined position;

generating second position information of a surgical component with respect to the surgical imaging device based on first image information obtained in a registration mode from the surgical imaging device;

determining the position of a surgical component with respect to the predetermined position based on first position information and the second position information; and

in an imaging mode, obtaining second image information from the medical imaging device of the surgical component based on the determined position.

(14)

The medical image processing method according to (13), wherein the position determination is further performed by determining the first position information indicating a position of the medical imaging device with respect to the predetermined position based on the arm position information and by determining the second position information from a stereoscopic distance between the patient and the medical imaging device.

(15)

The medical image processing method according to (13) to (14), wherein the registration mode or the imaging mode is activated based on the position information.

(16)

The medical image processing method according to (13) to (15), wherein the first image information is obtained in the registration mode at a different perspective that the second image information that is obtained in the imaging mode.

(17)

The medical image processing method according to (13) to (16), wherein the generating of the second position information of the surgical component is further performed by setting the position of the surgical imaging device as a reference point.

(18)

The medical image processing method according to (14), wherein the first position information of the surgical imaging device is based on arm position information from a supporting arm having attached thereto the surgical imaging device, and wherein the arm position information includes information of movement of at least one joint in the supporting arm.

(19)

The medical image processing method according to (18), wherein the information of movement of at least one joint in the supporting arm includes an amount of rotation of each joint.

(20)

The medical image processing method according to (13) to (19), wherein the second position information is further generated by processing images of the surgical component obtained by the surgical imaging device as the first image information.

(21)

The medical image processing method according to (20), wherein the processing of the images of the surgical component obtained by the surgical imaging device is based on the focus points of the images.

(22)

The medical image processing method according to (13) to (21), wherein the position of the surgical component being a reference point for image registration between a previously obtained medical image and images obtained by the surgical imaging device as the second image information.

(23)

The medical image processing method according to (13) to (22), wherein the position of the surgical component being a reference point for superimposing at least one preoperative image on images obtained by the surgical imaging device as the second image information.

(24)

A surgical information processing apparatus, including:

a surgical imaging device configured to obtain images of a patient;

a supporting arm having attached thereto the surgical imaging device; and

the surgical information processing apparatus according to claim 1.

(25)

The surgical information processing apparatus according to (24), wherein the medical imaging device is a surgical microscope or a surgical exoscope.

(26)

The surgical information processing apparatus according to (24) to (25), wherein the supporting arm has an actuator at a joint.

(27)

A non-transitory computer readable medium having stored therein a program that when executed by a computer including circuitry causes the computer to implement a surgical information processing method implemented using circuitry, including:

obtaining first position information of a surgical imaging device, the first position information indicating displacement of the surgical imaging device from a predetermined position;

generating second position information of a surgical component with respect to the surgical imaging device based on first image information obtained in a registration mode from the surgical imaging device;

determining the position of a surgical component with respect to the predetermined position based on first position information and the second position information; and

in an imaging mode, obtaining second image information from the medical imaging device of the surgical component based on the determined position.

Additionally, the present technology may also be configured as below.

(1A)

A medical imaging apparatus including:

an arm posture information detection unit configured to detect posture information concerning a posture of an arm that includes at least one joint unit and supports a camera;

a camera information detection unit configured to detect information outputted from the camera; and

a position calculation unit configured to calculate a predetermined position on the basis of the posture information and the information outputted from the camera.

(2A)

The medical imaging apparatus according to (1A),

wherein the arm is fixed to a support base configured to support a patient and

the position calculation unit calculates a relative position to a predetermined reference position of which the position does not change even when the posture of the arm changes.

(3A)

The medical imaging apparatus according to (1A),

wherein the arm is mounted on a movable cart, and

the position calculation unit sets, as a reference position, a predetermined position specified on the basis of a reference marker fixed to a support base configured to support a patient and calculates a relative position to the reference position, in a state where the movable cart is placed in a predetermined position.

(4A)

The medical imaging apparatus according to (3A), further including:

an arm control unit configured to control the arm,

wherein, when a relative position of the reference marker at the time when a current reference position is set and a relative position of the reference marker calculated are different, the arm control unit corrects the posture information of the arm, with the calculated relative position of the reference marker as a reference.

(5A)

The medical imaging apparatus according to any one of (1A) to (4A), wherein the position calculation unit determines whether a predetermined object to be detected is present in an image captured by the camera or not, and calculates a position of the object to be detected in a case where the object to be detected is present.

(6A)

The medical imaging apparatus according to (5A), wherein the position calculation unit expands an imaging range of the image in a case where the predetermined object to be detected is not present in the image captured by the camera.

(7A)

The medical imaging apparatus according to any one of (1A) to (6A), further including an arm control unit configured to control the arm,

wherein the arm control unit registers a surgical site of a patient included in an image captured by the camera with a reference image prepared in advance by controlling the posture of the arm.

(8A)

The medical imaging apparatus according to (7A), wherein,

when the surgical site and the reference image are out of registration even when the registration is performed,

the arm control unit performs registration between the surgical site and the reference image again by adjusting a position of the camera, using a position of a virtual center of the surgical site as a pivot point.

(9A)

The medical imaging apparatus according to any one of (1A) to (8A), wherein the predetermined position is information indicating at least one of a focal distance of the camera, a position of a surgical site of a patient, a position of a surgical instrument, a position of a tip of a surgical instrument, and a position of a reference marker.

(10A)

The medical imaging apparatus according to any one of (1A) to (9A), wherein the arm posture information detection unit detects the posture information on the basis of an output of an encoder provided in the joint unit.

(11A)

The medical imaging apparatus according to any one of (1A) to (10A), wherein the information outputted from the camera includes one of information of a focal distance of the camera and an image signal acquired by the camera.

(12A)

The medical imaging apparatus according to any one of (1A) to (11A), further including:

an output unit configured to output 3D image information produced from an image signal acquired by the camera.

(13A)

A surgical navigation system including:

an arm posture information detection unit configured to detect posture information concerning a posture of an arm that includes at least one joint unit and supports a camera;

a camera information detection unit configured to detect information outputted from the camera;

a position calculation unit configured to calculate a predetermined position on the basis of the posture information and the information outputted from the camera;

an output unit configured to output 3D image information produced from an image signal acquired by the camera; and

a navigation control unit configured to perform navigation of an operation while causing an image in which a surgical site of a patient included in the 3D image information produced from the image signal is superimposed on a reference image prepared in advance to be displayed.

### REFERENCE SIGNS LIST

10, 10A imaging apparatus
14 microscope unit
14A stereo camera
30 arm unit
48 probe (surgical instrument)
50 navigation apparatus
54 display device
60 navigation control device
100 control device
110 position computation unit
112 arm posture information detection unit
114 camera information detection unit
116 position calculation unit
120 arm posture control unit
130 surgical instrument marker
134 reference marker

1. A surgical information processing apparatus, comprising:
circuitry configured to
obtain position information of a surgical imaging device, the position information indicating displacement of the surgical imaging device from a predetermined position,
in a registration mode, obtain first image information from the surgical imaging device regarding a position of a surgical component,
determine the position of the surgical component based on the first image information and the position information, and
in an imaging mode, obtain second image information from the surgical imaging device of the surgical component based on the determined position.

2. The surgical information processing apparatus according to claim 1, wherein the position determination is further performed by determining a position of the surgical imaging device with respect to the predetermined position based on the position information and by determining a distance between the surgical component and the surgical imaging device.

3. The surgical information processing apparatus according to claim 1, wherein the surgical component is one of a surgical site and a surgical instrument.

4. The surgical information processing apparatus according to claim 1, wherein the circuitry activates the registration mode or the imaging mode based on the position information.

5. The surgical information processing apparatus according to claim 1, wherein the first image information is obtained in the registration mode at a different perspective that the second image information that is obtained in the imaging mode.

6. The surgical information processing apparatus according to claim 1, wherein the position determination is further performed by setting the position of the surgical imaging device as a reference point.

7. The surgical information processing apparatus according to claim 1, wherein the position information of the surgical imaging device is based on arm position information from a supporting arm having attached thereto the surgical imaging device, and
wherein the arm position information includes information of movement of at least one joint in the supporting arm.

8. The surgical information processing apparatus according to claim 7, wherein the information of movement of at least one joint in the supporting arm includes an amount of rotation of each joint.

9. The surgical information processing apparatus according to claim 1, wherein the position determination is further performed by processing images of the surgical component obtained by the surgical imaging device as the first image information.

10. The surgical information processing apparatus according to claim 9, wherein the processing of the images of the surgical component obtained by the surgical imaging device is based on the focus points of the images.

11. The surgical information processing apparatus according to claim 1, wherein the position of the surgical component being a reference point for image registration between a previously obtained medical image and images obtained by the surgical imaging device as the second image information.

12. The surgical information processing apparatus according to claim 1, wherein the position of the surgical component being a reference point for superimposing at least one pre-operative image on images obtained by the surgical imaging device as the second image information.

13. A surgical information processing method implemented using circuitry, comprising:
obtaining first position information of a surgical imaging device, the first position information indicating displacement of the surgical imaging device from a predetermined position;
generating second position information of a surgical component with respect to the surgical imaging device based on first image information obtained in a registration mode from the surgical imaging device;
determining the position of a surgical component with respect to the predetermined position based on first position information and the second position information; and
in an imaging mode, obtaining second image information from the medical imaging device of the surgical component based on the determined position.

14. The medical image processing method according to claim 13, wherein the position determination is further performed by determining the first position information indicating a position of the medical imaging device with respect to the predetermined position based on the arm position information and by determining the second position information from a stereoscopic distance between the patient and the medical imaging device.

15. The medical image processing method according to claim 13, wherein the registration mode or the imaging mode is activated based on the position information.

16. The medical image processing method according to claim 13, wherein the first image information is obtained in the registration mode at a different perspective that the second image information that is obtained in the imaging mode.

17. The medical image processing method according to claim 13, wherein the generating of the second position information of the surgical component is further performed by setting the position of the surgical imaging device as a reference point.

18. The medical image processing method according to claim 14, wherein the first position information of the surgical imaging device is based on arm position information from a supporting arm having attached thereto the surgical imaging device, and
wherein the arm position information includes information of movement of at least one joint in the supporting arm.

19. The medical image processing method according to claim 18, wherein the information of movement of at least one joint in the supporting arm includes an amount of rotation of each joint.

20. The medical image processing method according to claim 13, wherein the second position information is further generated by processing images of the surgical component obtained by the surgical imaging device as the first image information.

21. The medical image processing method according to claim 20, wherein the processing of the images of the surgical component obtained by the surgical imaging device is based on the focus points of the images.

22. The medical image processing method according to claim 13, wherein the position of the surgical component being a reference point for image registration between a previously obtained medical image and images obtained by the surgical imaging device as the second image information.

23. The medical image processing method according to claim 13, wherein the position of the surgical component being a reference point for superimposing at least one pre-operative image on images obtained by the surgical imaging device as the second image information.

24. A surgical information processing apparatus, comprising:
a surgical imaging device configured to obtain images of a patient;
a supporting arm having attached thereto the surgical imaging device; and
the surgical information processing apparatus according to claim 1.

25. The surgical information processing apparatus according to claim 24, wherein the medical imaging device is a surgical microscope or a surgical exoscope.

26. The surgical information processing apparatus according to claim 24, wherein the supporting arm has an actuator at a joint.

27. A non-transitory computer readable medium having stored therein a program that when executed by a computer including circuitry causes the computer to implement a surgical information processing method implemented using circuitry, comprising:
obtaining first position information of a surgical imaging device, the first position information indicating displacement of the surgical imaging device from a predetermined position;
generating second position information of a surgical component with respect to the surgical imaging device based on first image information obtained in a registration mode from the surgical imaging device;
determining the position of a surgical component with respect to the predetermined position based on first position information and the second position information; and
in an imaging mode, obtaining second image information from the medical imaging device of the surgical component based on the determined position.
